SEQUENCE LISTING (19 sequences)

SEQ ID NO: 1 - DNA, 1341 bp, Lactococcus lactis
atgaaaatcgtagttatcggtacaaaccacgcaggcattgctacagcgaatacattacttgaacaatatcccgggcatgaaattgtcatgattgaccgtaatagcaacatgagttatctaggttgtggcacagcaatttgggttggaagacaaattgaaaaaccagatgaattattttatgccaaagcagaggattttgaggcaaaaggggtaaaaattttgactgaaacagaagtttcagaaattgattttgctaataagaaagtttatgcaaaaactaaatctgatgatgaaataattgaagcttacgacaagcttgttttagcaacaggttcacgtccaattattcctaatctaccaggcaaagaccttaagggaattcattttctgaaactttttcaagaaggtcaagcaattgacgcagaatttgccaaagaaaaagtcaagcgtatcgcagtcattggtgcaggatatatcggtacagagattgcggaagcagctaaacgtcggggtaaagaagttcttctctttgacgctgaaaatacttcacttgcatcatattatgatgaagaatttgccaaaggaatggatgaaaaccttgctcaacatggaattgaacttcattttggagaactggccaaagaatttaaagcgaatgaggaaggttatgtatcacaaatcgtaaccaacaaggcgacttatgatgttgatcttgtcatcaattgtattggttttactgccaacagtgccttggcaagtgataagttagctaccttcaaaaatggcgcaatcaaggtggataagcatcaacaaagtagtgatccagatgtttacgcggtaggtgatgttgcgacaatttattctaatgccttgcaagattttacttatatcgctcttgcctcaaacgctgttcggtcaggaattgtcgcaggacacaatattggtggaaaagaattagaatctgttggtgttcaaggttctaatggtatttcgatttttggttacaatatgacttctacaggactttctgttaaagctgctaaaaaattaggtttagaagtttcatttagtgattttgaagataaacaaaaagcttggtttcttcatgaaaacaacgatagtgtgaaaattcgtatcgtatatgagacaaaaagtcgcagaattattggagcacaacttgctagtaaaagtgagataattgcaggaaatataaatatgttcagtttagcgattcaagagaaaaaaacaattgatgaactagctttgcttgatttattctttctcccccacttcaacagtccatataattatatgacagttgcagctttgaatgccaaataa

SEQ ID NO: 2 - DNA, 1500 bp, Enterococcus faecalis
aagcttgattcctctgatatattttaaacagacagagggagacgtcacctctgcaaaacaaagattgtgaaagaagggatttacaacatgaaagtcgtagtcgtaggatgtacccatgctggaacttcagcagttaaaagtattttagcaaatcacccggaggcggaagtcactgtatatgaaagaaatgacaatatttccttcctatcttgcggaattgccttgtacgttggaggcgtagttaaaaatgctgctgatttattttattcaaatccagaagaactggcttctttaggagcaacagtgaagatggaacacaatgttgaagaaatcaacgtggacgacaaaactgttacagcgaaaaacttacaaacgggcgcaactgagaccgtttcttatgataaattagtgatgacaactggttcttggccaattattccaccaattccaggaattgatgctgaaaatattttattatgtaagaactattcacaagccaacgtgatcatcgaaaaagcgaaagatgccaaacgtgtcgttgtcgttggtggcggttatattggtattgaattagtagaagcttttgtagaatcaggtaaacaagttactttagttgacgggttagaccgcattttaaataaatatttagataagccattcacagatgttctagaaaaagaattagttgatcgtggcgttaatttagctttaggtgaaaatgtccaacaattcgtagcagatgagcaaggaaaagtagcaaaagtcattacaccaagtcaagaattcgaagcagatatggtgattatgtgtgttggtttccgtccaaatacagaattattaaaagataaagttgacatgttaccaaatggtgcaatcgaagtcaatgagtatatgcaaacaagcaatcctgatatttttgctgcgggtgatagtgcagttgttcactacaacccaagtcaaacaaaaaattatatcccattagcaacgaatgctgttcgtcaaggtatgctagtaggtcgtaatttaactgaacaaaaattggcttatcgtggcacacagggaacttctggtttatatttattcggttggaaaattggttcaacaggtgtaacaaaagaaagtgccaaattaaacggactagatgtcgaagcaactgttttcgaagataactatcgtccagagtttatgccaacaactgaaaaagttctgatggaattagtctatgaaaaaggaacacaacgcattgttggtggtcaattgatgtctaaatatgatatcacacaatctgccaatactttatcattagcggtccaaaataaaatgactgtcgaagatttagcgatttctgatttcttcttccaaccacattttgatcgcccttggaactacttaaacttattagcccaagccgctttagaaaatatgtaattaataataatagaaaaaggatgaagaacttctgttcttcatcctttttgttagtcactaagcgctccagtt

SEQ ID NO: 3 - DNA, 1437 bp, Mycoplasma genitalium
ctattttttaggaatgtaatagctaaaaccaagtgcttgtaaaacagttgctaacacaaagttaaacggtttgttgtaatgtggaagaaaataaacatccactaaacccagttcagttagtaacatctgcttttggattgcaagtgcaatatagaaaataatctcactgtgattggtattccaagaaaggagttgtgctcctaaaattcttagggttttcttgtcatatacaagtttaaaacgcaccttgtcataactgcccataaactcaggacgatcattgtcatcaacaactgatatgccaacatcaaaacctaacttcttagcacgctgttcagttaatccacatgctgctaaatttaaaccaaagatatggagtgcattggtgccaacgatagattggagtttaacttggttactaccaatgatatgcatcgcagctactaatccactctttactgcattggtagcaagatcgatgttttcatactgttcactagcagcattgtaaatagcagcacaacccccaatgacataaacatccttatgatttagtgcttggagaaattcattaactttaattgaaccgttgtgaataaactcaaagttttgatctttaggaacaaacttagtgctaggtctaaacccaattgattggttcacaaggtctgcatttactattcccttatcagtttcaactcctttgacaacgttgtttgtactatcaacaacaaagcccttaacactgcaacccatcattagttttaacccatctttttgcatcactttttcaagttcatcagtaaactcatgatcaaagttattaccagcaggcttatcaagtaagtcaattactgttacttgctttttgcataaccaagctgcttcagcaagttccaagccaatgtaaccagaaccaacaatagcaactgatttaatggttttatctttacgaaaactatcgattaaggtaagtgcatgttggtataacttacagctaattaagttcttaacattaccacaatatttgtcagtgtagttaaactccaaaggcttgtgtgtcaccttgttttcaacattcatacatataggtcatgctcctgaagcgattactagttgatcaaactgatcagtgaactctttatttgatgttaaatctctaactgttacctgttttttgattagatcaatgttagtaacatcatgactcataaagatgttagcgcccatctgtttcaactcctcagggttggaatagaaaagatcatcagtgtttttaacaacaccactaacagcaagtgcaattccacaccccagaaacgagatgtttgtgtttctatcataagcgttaaccttaaagtccttactttttgaaagtaaagttctaataaaactagtaccagcgtgatttattccaatcacaatcacttttttcat

SEQ ID NO: 4 - DNA, 1800 bp, Streptococcus mutans
ttaattatattaaatcaaaataaaaatttaaaatcactcgatatacaatcttgtttgaatagctcttgaaagctagcaatcaggaacttatatattgagtgattttttgattccttatcaattgtaaattatagtagagaagtccattttggagattataaagttatttttatgaaagatgagcatattctttgtaagcgtttcaacctcatgctatactaatgaagtaataattagattatattgaaaagaggatttgcttatgagtaaaatcgttattgttggagctaaccatgcaggtacagctgccattaatactattctagataattacggtagtgaaaacgaagttgtcgtttttgaccaaaattctaatatttcattcttgggttgtggaatggcactttggattggaaaacaaatatcaggccctcaaggtcttttttatgctgacaaggaatcgttagaagcaaaaggtgctaaaatttatatggaatcgccagtgacagccattgattatgatgctaagagggttactgctttggtcaatggtcaagaacatgttgaaagctatgagaagcttattttggcaacaggatcaacaccaatcttaccacctatcaaaggtgcagctatcaaagaaggtagtcgtgattttgaagcaactttgaaaaatcttcaatttgttaaattgtatcaaaatgcagaagatgttattaataaattacaggataagagtcaaaatctgaatcgtattgctgttgttggtgctggttatattggtgtagaacttgctgaagcctttaaacgcctcggaaaagaagtgattcttattgatgttgttgatacttgcttagctggttattatgatcaggatctttcagaaatgatgcgtcaaaatttggaagatcatggtattgaattagcattcggagaaactgtcaaagccattgaaggtgatggtaaagtcgaacgtattgtaactgataaagcgagccatgatgtggatatggttattttagctgtcggtttccgtcctaatactgcacttggcaacgctaaactcaaaaccttccgtaatggtgctttccttgttgataaaaaacaagagacaagtattcctgacgtttatgccatcggcgattgcgcgactgtttatgacaacgctattaatgataccaattatattgccttagcttcaaacgctcttcgctcaggtattgtagctggtcataatgcagcagggcataaattggaatctcttggtgttcaaggttcaaatggtatttcaatttttggtctcaatatggtttcaactgggttaacacaagaaaaagcaaagcgttttggctataatccagaagtcactgcatttacagattttcagaaggctagttttattgaacatgataattatcctgttacacttaaaattgtctatgataaggatagccgactggttcttggtgcacaaatggcatctaaagaagatatgtcaatgggaattcatatgttttcattggctattcaggaaaaagttaccattgaacgtttagctctactggactatttctttcttcctcatttcaatcaaccctataattatatgaccaaagcagcattaaaagctaaatgattatgaatgaaacagtgaaaagtaattttttgtcagtttaatcgtttttcaagaaaactattgagttatctttatctatcaaattctcatacttttatcattaggtatattgctgtctgaaagtgattattatataataaaagtgaaaaggaggcgttagaaag

SEQ ID NO: 5 - DNA, 1440 bp, Mycoplasma pneumoniae
atgaagaaagtgattgttatcggtgtcaatcacgccggcaccagttttatcagaacccttttaagcaagagcaaagactttcaggtcaacgcttatgatcgcaataccaacatctcctttttgggttgtggaattgccttggcagttagtggtgtagttaaaaacaccgaagacctgttctactccacccctgaagagctaaaagctatgggtgctaatgtctttatggctcatgatgtagttggtcttgatctagacaaaaaacaagtaatcgtgaaggacttggccactggtaaggaaactgtggaccactatgatcaattggtagtagcctctggagcttgaccgatttgtatgaatgtggaaaacgaagtaacccacacccaattgcaattcaaccacactgataagtactgtggcaatattaagaacttaattagttgtaagttgtaccaacacgcccttaccttaattgacagcttccgtcatgacaagtcaattaaatcagtagccattgtgggctcgggttacattggtttagagttagcagaagcggcttgacagtgtggtaaacaggtgactgtgattgacatgttggacaaaccagctggcaataactttgatgaagagtttaccaacgagttggaaaaagccatgaagaaagcgggcattaacctaatgatgggtagtgccgttaagggctttattgttgatgcggacaaaaatgtcgttaaaggtgttgaaaccgataagggtcgggtggatgctgaccttgtcatccaatcgattgggttccgcccaaacacccagtttgtccctaaagacaggcagtttgagtttaaccgtaacggttcgattaaggttaatgaatacctccaagccttaaatcatgaaaatgtgtatgttattggtggggctgctgccatttatgatgcagctagtgaacaatacgaaaacattgacttagctaccaacgctgtgaaaagtggtttagtcgctgctatgcacatgattggtagcaaggctgtaaagttagagtccattgttggtaccaatgcgctgcacgtatttggtttaaatctagcagctactggtttaaccgaaaaacgtgccaaaatgaacggttttgatgtgggagtgagcattgttgatgacaacgaccgtcccgaatttatggggacctttgacaaggtgcgctttaagttaatttacgacaagaagacgctccgtttgttgggtgcccaattactttcgtgaaacaccaaccacagtgaaattatcttctatattgctttagcagtacagaagaaaatgttgatcagtgaactgggtttagtagacgtttactttcttccccactacaataagccgttcaactttgtgttagcagccgtgttgcaagcgcttggttttagttactatacccctaaaaataaataa

SEQ ID NO: 6 - DNA, 1392 bp, Methanococcus japanicus
ttggttaatagaaaaccaaataaccccaataaaaatggtgaggaaatgagagcaataataataggaagtggagctgctggtttaacaacagcatcaacaatcagaaaatacaacaaagatatggaaatagtagtaataacaaaagaaaaggaaatagcttattctccatgtgcaattccctatgttattgagggagcaataaagagctttgatgacattattatgcacactccagaagattacaaaagagagagaaacatcgatatattaactgaaactaccgttatagatgttgattcaaaaaataacaaaataaagtgtgtagataaagatggaaatgagtttgagatgaattatgattacttagttttagcaactggagcagagccatttatccctccaattgaaggaaaagacttagatggagtatttaaagttagaactattgaggatggtagggctatattaaaatacattgaagaaaatggctgtaaaaaagttgctgttgttggagctggagctattggcttggagatggcttatggtttaaaatgtagaggtttagatgtcttagttgttgagatggctcctcaagtgttaccaagattcttagacccagatatggctgagatagttcaaaaatatttagaaaaagaaggaattaaggttatgctatcaaaaccattggaaaagattgttggaaaagagaaggttgaagcagtttatgttgatggtaagttgtatgatgttgatatggttattatggctactggtgtaaggccaaatattgagttggctaaaaaagctggctgtaaaattggaaaatttgcaatagaagtaaatgagaagatgcaaacctctataccaaacatctatgcagttggagattgtgttgaagttattgactttataactggagagaaaacactatctccatttggaactgctgctgtaaggcaaggaaaagttgctggtaaaaatatagctggagttgaagcaaagttctatccagttttaaactctgctgttagcaaaataggagatttagagattggaggaacaggtttaacagcattctctgccaatttaaagagaattccaatagttattggtagagctaaggcgttaacaagagcaagatactatccaggaggaaaagagattgagataaagatgatatttaatgaagatggcaaagtagttggatgtcaaatcgttggtggagagagagttgctgaaagaatagatgcaatgtctatagcaatatttaaaaaagttagtgcagaggaacttgcaaatatggagttctgctatgctccaccagtttctatggtgcatgagccattatcattagctgctgaagatgctttgaaaaagttgagtaataaataa

SEQ ID NO: 7 - DNA, 32 bp, Artificial sequence
  primer
cggcgctcgagatgaaaatcgtagttatcggt

SEQ ID NO: 8 - DNA, 32 bp, Artificial sequence
  primer
cggcgtctagattatttggcattcaaagctgc

SEQ ID NO: 9 - DNA, 29 bp, Artificial sequence
  primer
cggagctccagttcgagtttatcattatc

SEQ ID NO: 10 - DNA, 28 bp, Artificial sequence
  primer
cgggatcctcgaaactaagttcttggtg

SEQ ID NO: 11 - DNA, 29 bp, Artificial sequence
  primer
cgggatccatgaaaatcgtagttatcggt

SEQ ID NO: 12 - DNA, 29 bp, Artificial sequence
  primer
cgctcgagttatttggcattcaaagctgc

SEQ ID NO: 13 - DNA, 30 bp, Artificial sequence
  primer
ggcggcatgcgctccagttcgagtttatca

SEQ ID NO: 14 - DNA, 31 bp, Artificial sequence
  primer
cggcggcatgctttcacacaggaaacagcta

SEQ ID NO: 15 - DNA, 23 bp, Artificial sequence
  primer
atcccccgggctgcaggtcgacc

SEQ ID NO: 16 - DNA, 31 bp, Artificial sequence
  primer
cggcggatatcgctccagttcgagtttatca

SEQ ID NO: 17 - DNA, 31 bp, Artificial sequence
  primer
cggcgactagttttcacacaggaaacagcta

SEQ ID NO: 18 - DNA, 60 bp, Artificial sequence
  primer
tgattcggtaatctccgagcagaaggaagaacgaaggaaggcaggtcgacaacccttaat

SEQ ID NO: 19 - DNA, 59 bp, Artificial sequence
  primer
tgagtttagtatacatgcatttacttataatacagtttttttcacacaggaaacagcta

(19) United States
(12) Patent Application Publication
(10) Pub. No.: US 2010/0047387 A1
(43) Pub. Date: Feb. 25, 2010

(54) TRANSFORMED SACCHAROMYCES YEAST STRAINS HAVING REDUCED ETHANOL PRODUCTION BY FERMENTATION

(76) Inventors: Stephanie Heux, Echandelys (FR); Jean-Marie Sablayrolles, Montpellier (FR); Sylvie Dequin, Montpellier (FR)

Correspondence Address: STITES & HARBISON PLLC, 1199 NORTH FAIRFAX STREET, SUITE 900, ALEXANDRIA, VA, 22314, US

(21) Appl. No.: 11/917,681

(22) PCT Filed: Jun. 15, 2006
(86) PCT No.: PCT/FR06/01364

§ 371 Date: Jun. 30, 2008

(51) Int. Cl. C12N 1/19; C12N 15/74; C12N 15/63; C12G 1/00; C12G 3/00; A21D 8/00

(52) U.S. Cl. 426/15; 435/254.21; 435/471; 435/320.1; 426/11; 426/27

(57) ABSTRACT

The invention concerns transformed yeast strains belonging to the genus Saccharomyces comprising a heterologous nucleic acid encoding an NADH oxidase producing water, and a method for preparing same, and their use in fermenting methods for transforming sugars with a reduced ethanol production relative to non-transformed wild Saccharomyces strains.

The present invention relates to transformed yeast strains belonging to the genus Saccharomyces which comprise a heterologous nucleic acid coding for a water-producing NADH oxidase, and to the use thereof in fermentation processes for converting sugars with a reduced ethanol production yield as compared with non-transformed wild-type Saccharomyces strains.

For about fifteen years, scientific knowledge and know-how in viticulture and enology have led to a very significant improvement in the organoleptic qualities of wines. Current wine-growing practices favour the production of wines having a high qualitative potential by delaying grape gathering. A major consequence is the increase in the sugar content of the musts, and therefore in the alcohol content of the wines (frequently greater than 14°). This trend, which is encountered in most production areas, openly presents a number of problems for the world wine-making field, because excessive alcohol contents are not readily compatible with consumers' concerns regarding their health and well-being and, in addition, are subject to taxes in some countries.

Accordingly, there is an increasing demand for methods and tools which enable the alcohol content of wines to be reduced. Physical approaches (for example vacuum distillation) are increasingly being used, but they are not readily compatible with the maintenance of a satisfactory organoleptic quality.

A biological solution would be based on the use of yeast strains with a low alcohol yield.

For example, S. cerevisiae yeasts, especially enological S. cerevisiae yeasts, convert the sugars into alcohol with a yield of 0.47 g/g, which varies little depending on the strain used. Accordingly, in order to obtain a S. cerevisiae yeast having a low alcohol yield, it is necessary to employ genetic strategies aimed at diverting some of the sugars to the formation of other by-products.

Several genetic engineering approaches have been employed, some by the inventors, for diverting some of the sugars to the production of by-products other than ethanol (Dequin, 2001; Dequin et al., 2003). These approaches were based on modifying the activity of enzymes involved in the synthesis of glycerol or in the use of pyruvate. For example, overproduction of glycerol, obtained by overexpression of GPD1 or GPD2 coding for glycerol 3-phosphate dehydrogenase (Nevoigt and Stahl 1996; International Patent Application WO 96/41888; Michnick et al., 1997; Remize et al., 1999; Remize et al., 2000; Remize et al., 2001; de Barros Lopez et al., 2000; Eglinton et al., 2002), thus enabled the ethanol content to be reduced by 1 to 2°. However, it is accompanied by major modifications of the level of production of other metabolites, some of which are undesirable in wine. Likewise, it is possible to divert some of the sugars to the production of lactic acid by expressing a bacterial lacticodehydrogenase. However, the alcohol content cannot be lowered significantly given the lactic acid contents acceptable in wine (International Patent Application WO 94/00554; Dequin and Barre, 1994). Another strategy has been developed, based on the expression of a glucose oxidase, which would permit the oxidation of some of the sugars to gluconic acid in the presence of oxygen (Malherbe et al., 2003). It is probable, however, that the level of gluconic acid production required to significantly lower the ethanol content is incompatible with the maintenance of the organoleptic qualities of the wine.

There is, therefore, still a need for yeast strains having a low alcohol yield that can be used in practice in the field of enology.

However, genetic engineering approaches come up against the difficulty of successfully identifying the enzymatic activity or activities to be modified in order to achieve the expected metabolic changes.

The possibility of inducing a modification of the metabolic fluxes by modifying the level of oxidation of the NADH has previously been demonstrated in the bacterium Lactococcus lactis. It has thus been observed that the overexpression of the NADH oxidase of Streptococcus mutans in Lactococcus lactis reorients the metabolism towards the formation of acetoin or of diacetyl at the expense of lactic acid (Lopez de Felipe and Hugenholtz, 1999; Lopez de Felipe et al., 1998).

However, unlike bacteria, yeasts do not possess NADH oxidase. The inventors have shown that the introduction into a Saccharomyces yeast of a heterologous gene coding for a water-producing NADH oxidase induces a modification of the metabolism of ethanol.

The invention therefore relates to a Saccharomyces yeast transformed with a heterologous gene coding for a water-producing NADH oxidase, and to the use thereof, especially in enology.

### DEFINITIONS

Within the context of the present invention, the term “yeast” denotes a yeast of the genus Saccharomyces. Said yeast can be selected, for example, from one of the following species: Saccharomyces bayanus, Saccharomyces carlsbergensis, Saccharomyces cerevisiae, Saccharomyces paradoxus, Saccharomyces pastorianus, and Saccharomyces uvarum. The Saccharomyces yeast according to the invention is preferably Saccharomyces cerevisiae (S. cerevisiae). The expression “enological strain” is understood as meaning a S. cerevisiae strain. A very large number of enological S. cerevisiae strains are commercially available or have been described in the prior art. Some commercially available enological strains are S. bayanus var. uvarum. A “coding sequence” or sequence “coding for” an expression product, such as a RNA, a polypeptide, a protein or an enzyme, is a nucleotide sequence which, when expressed, results in the production of that RNA, polypeptide, protein or enzyme. A sequence coding for a protein can include a start codon and a stop codon.

The term “heterologous” refers to a combination of elements that does not occur naturally. Within the context of the present invention, a heterologous nucleic acid sequence refers to a nucleic acid sequence (gene, cDNA or RNA) which is not naturally present in the cell, that is to say a sequence that is foreign or exogenous to the cell.

The expression “water-producing NADH oxidase” or “Nox, H₂O” is understood as meaning an enzyme which catalyzes the reaction: 2NADH+2H⁺+O₂→2NAD⁺+2H₂O. It can be in particular a bacterial enzyme. A number of water-producing NADH oxidases have in fact been identified in bacteria and have been recorded especially in Table 1 of the article of Riebel et al., 2002.

A heterologous nucleic acid coding for a water-producing NADH oxidase can, for example, be constituted by, or comprise, a coding sequence selected from the group constituted by the genes of the enzymes Nox, H₂O identified in Lactococcus lactis (Hoefnagel et al., 2002; accession number Genbank AY046926; SEQ ID No.1), Enterococcus faecalis (Ross and Clairbone, 1992; accession number Genbank X68847; SEQ ID No.2), Mycoplasma genitalis (Peterson et al., 1993; accession number Genbank U39707; SEQ ID No.3), Streptococcus mutans (Matsumoto et al., 1996; accession number Embl 815515; SEQ ID No.4), Mycoplasma pneumoniae (Himmelreich et al., 1996; accession number Embl MPAE44, SEQ ID No.5), Methanococcus japanicus (Bult et al., 1996; accession number Embl MJU67512, SEQ ID No.6), and Leuconostoc mesenteroides (Koike et al., 1985).

In addition to the actual sequence coding for the enzyme Nox, H₂O, the heterologous nucleic acid coding for a water-producing NADH oxidase can comprise regulation or control sequences, such as a start codon, a stop codon, a promoter, a signal or secretion sequence or other sequences used by the genetic machinery of yeasts.

The expression “alcoholic fermentation” denotes the sequence of reactions for the conversion of pyruvate into ethanol and, by extension, the totality of the reactions for the conversion of sugars into ethanol.

Transformed Saccharomyces Yeasts and Preparation Method or Process

The introduction into a Saccharomyces yeast of a heterologous gene coding for a water-producing NADH oxidase induces a modification of the metabolism of ethanol.

The inventors have, in fact, expressed in a S. cerevisiae strain the gene noxE, which codes for a water-forming NADH oxidase in Lactococcus lactis (Hoefnagel et al., 2002; Lopez de Felipe and Hugenholtz, 2001). Fermentations were carried out in a synthetic medium mimicking the composition of a grape must, with the supply of oxygen in order to permit functioning of the NADH oxidase.

Analysis of the strains obtained shows that the expression of the NADH oxidase induces a significant reduction in the production yield of ethanol. That reduction is accompanied by other metabolic changes, namely a reduction in the production of glycerol, of α-ketoglutarate and of hydroxyglutarate, and an accumulation of acetaldehyde as well as of acetate and acetoin.

The invention therefore relates to a transformed yeast strain belonging to the genus Saccharomyces which comprises a heterologous nucleic acid coding for a water-producing NADH oxidase.

The invention relates also to a method for the preparation of a transformed yeast strain belonging to the genus Saccharomyces which has, in alcoholic fermentation, a production yield of ethanol that is reduced as compared with the non-transformed wild-type Saccharomyces strain.

Said method comprises a step consisting of the transformation of a so-called “wild-type” yeast strain of the genus Saccharomyces by introducing at least one heterologous nucleic acid coding for a water-producing NADH oxidase.

The expression “transform” or “transformation” means the introduction of a gene or of a foreign (that is to say exogenous) RNA or DNA sequence into a host Saccharomyces yeast, so that the host yeast expresses the gene or sequence that has been introduced in order to produce the desired substance, in the present case a water-producing NADH oxidase enzyme. The yeast strain which has received and expresses the nucleic acid coding for a water-producing NADH oxidase has been “transformed”.

The sequence coding for the water-producing NADH oxidase can be “under the control of” or “associated in a functional manner with” transcription and/or translation control sequences so as to permit regulation of the expression of said coding sequence in the yeast. They can be sequences of the promoter and terminator type which are active in the yeast, for example promoters and terminators of the alcohol dehydrogenase 1 (ADH1), phosphoglycerate kinase (PGK) or glyceraldehyde-3-phosphate dehydrogenase (GAPDH) genes.

The association of the sequence coding for the water-producing NADH oxidase and sequences permitting regulation of its expression constitutes an “expression cassette”.

The invention therefore relates also to an expression cassette comprising a nucleic acid coding for a water-producing NADH oxidase, preferably of bacterial origin, associated with sequences for regulation of the expression of said sequence coding for a water-producing NADH oxidase in yeast.

The nucleic acid coding for the water-producing NADH oxidase, or a cassette containing it, can be carried by a vector or integrated into the genome (chromosomal DNA) of the transformed yeast.

A “vector” can be used to carry the nucleic acid sequence coding for the water-producing NADH oxidase into the host yeast, or the cassette containing it, in order to transform the yeast and facilitate the expression of the sequence that is introduced. It can be, for example, a DNA vector of the plasmid type. Transformation of yeasts generally uses “shuttle” vectors in which the nucleic acid sequence coding for the water-producing NADH oxidase can be combined with a sequence permitting its expression in yeast, such as a yeast promoter. Such shuttle vectors comprise other additional sequences for permitting expression in bacteria, such as E. coli, or in other microorganisms. Such additional sequences which do not originate from yeasts serve solely for the construction of the vectors.

The invention therefore relates further to a vector comprising an expression cassette comprising a nucleic acid coding for a water-producing NADH oxidase, preferably of bacterial origin, associated with sequences for regulation of the expression of said sequence coding for a water-producing NADH oxidase in yeast.

The DNA vector can be introduced by any technique known to the person skilled in the art, especially by transformation with lithium acetate, by electroporation or with the aid of protoplasts. For example, the transformation method using lithium acetate described by Schiestl and Gietz (1989) can be used.

Said yeast strain of the genus Saccharomyces is preferably an industrial strain used in enology, in brewing, in baking or in cider-making.

According to a preferred embodiment, said yeast strain of the genus Saccharomyces is a Saccharomyces cerevisiae strain, preferably the enological strain S. cerevisiae V5 (also called ScV5M, deposited on 18 Jun. 1992 at the Collection Nationale de Cultures de Microorganismes, held by the Institut Pasteur, under number 1-1222).

Said water-producing NADH oxidase is preferably a bacterial NADH oxidase. Said nucleic acid coding for a water-producing NADH oxidase can comprise a sequence selected from the group constituted by the sequences SEQ ID No.1, SEQ ID No.2, SEQ ID No.3, SEQ ID No.4, SEQ ID No.5 and SEQ ID No.6.

According to a preferred embodiment, said nucleic acid coding for a water-producing NADH oxidase preferably comprises the gene of the enzyme Nox, H₂O identified in Lactococcus lactis (SEQ ID No.1).

Advantageously, the production yield of ethanol, by alcoholic fermentation, of the transformed strain of the genus Saccharomyces, as compared with the wild-type strain of the genus Saccharomyces, can be reduced by from 10 to 20%, preferably by approximately 15%, which represents a reduction of at least 0.5°, preferably at least 1°, more preferably at least 2°, as a function of the initial sugar content of the fermentation medium.

The degree of alcohol represents the number of ml of pure ethyl alcohol contained in 100 ml of a liquid, measured at 20° C. 1° of alcohol corresponds to 7.80 g/l of ethanol.

### Use of the Transformed Saccharomyces Yeasts

The Saccharomyces yeast strains according to the invention are capable of converting the sugar from their culture medium while producing a smaller amount of ethanol as compared with the corresponding wild-type Saccharomyces yeasts.

Those yeast strains can therefore be used especially in processes for the preparation of fermented drinks in order to allow drinks to be obtained that have a reduced ethanol content as compared with drinks prepared by alcoholic fermentation with the aid of non-transformed Saccharomyces yeasts. Those yeasts can likewise be used in baking in bread-making processes.

The invention therefore relates to the use of a Saccharomyces yeast strain according to the invention for carrying out an alcoholic fermentation. Said alcoholic fermentation can be carried out in a bread-making, wine-making, brewing, cider-making or distilling process. A yeast strain according to the invention can therefore be used more particularly for the preparation of a fermented drink, such as wine, champagne, beer, cider or a brandy, or in bread-making, especially for the preparation of bread.

The invention relates also to an alcoholic fermentation process which permits the conversion of sugars with a reduced production yield of ethanol. Said process comprises steps consisting of:

a) inoculating a medium having a high sugar content with at least one Saccharomyces yeast strain according to the invention;

b) cultivating said Saccharomyces yeast strain and allowing the fermentation to take place in order to convert the sugars into alcohol.

The culture can be carried out with an oxygen supply (microoxygenation), which can be continuous or discontinuous, and wherein the oxygen is supplied under limiting or non-limiting conditions. When the culture is carried out with a continuous oxygen supply, the yeast strain according to the invention is preferably cultivated under non-limiting oxygen conditions, that is to say under conditions where oxygen remains dissolved in the medium and is not consumed by the yeasts.

The expression “medium having a high sugar content” is understood as meaning a medium comprising at least 30 g/l of sugars, preferably at least 50 g/l of sugars, more preferably at least 80 g/l of sugars.

The term “sugars” denotes glucides in general, and more specifically monosaccharides such as glucose or fructose, or polysaccharides such as sucrose or maltose, for example.

Said alcoholic fermentation process can be a brewing process. The medium having a high sugar content is then a beer wort prepared from a mixture of barley and hops.

Said alcoholic fermentation process can also be a bread-making process. The medium having a high sugar content is then the dough that is left to rise, for example a bread dough, a brioche dough, etc.

According to a preferred embodiment, said alcoholic fermentation process is a wine-making process. The medium having a high sugar content is then a grape must. The sugar content of the grape musts used in wine-making is generally from 140 to 260 g/l.

The yeasts most frequently used in wine-making are S. cerevisiae yeasts, which generally have a production yield of ethanol of the order of 0.47 g per g of sugars. The use of such yeasts in wine-making (under conventional conditions, that is to say under anaerobiosis) thus produces wines having an alcohol content of from 8 to 15°.

The inventors have shown that the transformed S. cerevisiae yeasts according to the invention have a conversion yield of sugars into alcohol of 0.39 g per g of sugars, which represents a reduction of the order of 15% as compared with the alcohol yield of the wild-type strains. The use of such yeasts can yield wines having an alcohol content of from 7 to 13°.

For example, the inventors have shown that it is possible by using a transformed S. cerevisiae yeast strain according to the invention for carrying out a fermentation under controlled oxygenation conditions, starting from a synthetic medium having a sugar content of 200 g/l, to obtain a reduction of 1° in the ethanol content as compared with the content which would have been obtained by fermentation under anaerobic conditions using the wild-type S. cerevisiae strain (in the region of 12°), or a reduction of 1° in the ethanol content as compared with the content which would have been obtained by fermentation with the wild-type S. cerevisiae strain under the same controlled oxygenation conditions.

It is preferable to carry out a controlled oxygenation during the fermentation process. The activity of the NADH oxidase at a high level from the start of the fermentation can in fact slow down the growth of the yeasts and reduce their ability to degrade high sugar concentrations (greater than 80 g/l). These effects appear to be linked to the reduction in effectiveness of the system of detoxication of acetaldehyde via butanediol dehydrogenase (BDH). However, it is possible to limit those effects by controlling the oxygen supply in order to supply it in a limited quantity.

On the other hand, those effects are eliminated completely if the growth phase of the yeasts is separated from the phase of activity of the NADH oxidase, in other words if the activity of the NADH oxidase only manifests itself at the end of the growth phase of the yeasts (stationary phase). Thus, when the oxygen is supplied in a discontinuous manner, only in the stationary phase, during which phase at least 70% of the sugars are consumed, the totality of the glucose is consumed. The increase in acetaldehyde and acetate linked to the use of the transformed yeasts according to the invention then remains very moderate.

The cultivated yeasts multiply until one of the substrates of the culture medium IS is exhausted. For example, when the culture medium is a grape must, or a synthetic medium mimicking grape must, nitrogen is present in the medium in a limiting quantity relative to the sugars (approximately from 300 to 500 mg/l of assimilable nitrogen against approximately 200 g/l of sugars). The fermentation occurs as soon as the multiplication phase of the yeasts starts and continues once the yeasts have stopped growing. For such fermentation media, the stopping of growth caused by exhaustion of nitrogen and micronutrients in the medium manifests itself when approximately 30% of the sugars have been consumed.

The invention proposes more particularly a wine-making process comprising steps consisting of:

a) inoculating a grape must with at least one Saccharomyces yeast strain according to the invention;

b) cultivating said Saccharomyces yeast strain and allowing the fermentation to take place in order to convert the sugars into alcohol;

in which process said Saccharomyces yeast strain is first cultivated under anaerobic conditions and is then cultivated under aerobic conditions when the stationary growth phase of the yeasts has been reached, especially when substantially the totality of the assimilable nitrogen in the grape must has been consumed.

The expression “aerobic conditions” is understood as meaning culture conditions in the presence of oxygen, preferably under non-limiting oxygen conditions for the cultivated Saccharomyces strain. For a given medium and a given Saccharomyces strain, the person skilled in the art is able to determine if non-limiting culture conditions are present simply by detecting the presence of dissolved oxygen in the culture medium. For example, for a V5 strain transformed with the noxE gene of L. lactis, cultivated on a synthetic medium mimicking a grape must as described in the Examples (MS medium comprising 180-200 g/l of glucose, 6 g/l of malic acid, 6 g/l of citric acid, 460 mg/l of nitrogen, in the form of NH₄Cl (120 mg/l) and of amino acids (340 mg/l)), an oxygen transfer of 10 mg/l/h corresponds to non-limiting microoxygenation conditions.

Said Saccharomyces yeast strain is preferably a S. cerevisiae strain, more preferably an enological S. cerevisiae strain such as the V5 strain.

The invention relates also to the use of a Saccharomyces yeast according to the invention for regenerating NAD+ from NADH, for example during biotransformations.

Biotransformation consists in using living organisms to effect reactions which are difficult to carry out by chemical laboratory methods. In general, the living organism is provided with one or more precursor molecules and, after a period of time sufficient for metabolism to occur, there is isolated from the culture medium or from the biomass a product or products which differ(s) from the precursor molecules by an enzymatic modification or by a small number of enzymatic modifications.

Many biotransformations used for the production of pure enantiomers (alcohols, hydroxy acids, amino acids, etc.) or other compounds for fine chemistry (green chemistry) involve reduction reactions which require an electron acceptor, typically a cofactor of the NADH or NADPH type. A major problem which restricts the use of biotransformations is the regeneration of NAD(P) from NAD(P)H. The coupling of the biotransformations with another enzymatic reaction, for example the reaction catalyzed by alcohol dehydrogenase or by lactate dehydrogenase, permits effective regeneration of the cofactors. However, problems remain to be solved in terms of implementation, stability of the enzyme and overall cost. Recently, it has been proposed to use NADH oxidases which catalyze the oxidation of NADH by reducing molecular oxygen to water (Riebel et al., 2002).

As compared with those approaches, the use of whole cells having an integrated system for regenerating the cofactors allows biotransformations to be carried out more simply and at a lower cost.

The Examples and Figures which follow illustrate the invention without limiting the scope thereof.

### FIGURES

FIG. 1 shows a reaction scheme of the metabolic pathway of glucose degradation.

FIG. 2 shows the structure of the expression plasmids used for expressing NADH oxidase NoxE in the yeast S. cerevisiae.

FIG. 3 shows the integration of the expression cassette TDH3p-noxE-PGKt at the locus URA3.

FIG. 4 shows the change in the residual glucose and the dissolved oxygen for the control strain V5 cultivated on MS medium with an oxygen supply of 10 mg/l/h.

FIG. 5 shows the impact of the expression of the oxidase on the growth, the glucose consumption and the metabolic profiles of the strains V5 and V5noxE cultivated on MS medium with a constant oxygen supply at 10 mg/l/h.

FIG. 6 shows the change in the residual glucose and the dissolved oxygen for the strains V5 and V5noxE in stationary phase with an oxygen supply of 10 mg/l/h.

FIG. 7 shows the measurement of the growth, the glucose consumption and the metabolic profiles of the strains V5 and V5noxE cultivated on MS medium with an oxygen supply at 10 mg/l/h in stationary phase.

### EXAMPLES

Example 1

### Preparation of Transformed Yeast Strains Expressing noxE

In order to obtain strong and constitutive expression of the NADH oxidase of L. lactis in the yeast, the coding region of the noxE gene was amplified from the genomic DNA of L. lactis MG1363 and placed under the control of yeast regulatory elements, either in yeast shuttle vectors I E. coli or integrated into the genome of the yeast.

1. Introduction of the NADH Oxidase on the Multicopy Plasmid pVT100-U ZEO Under the Control of the ADH1 Promoter

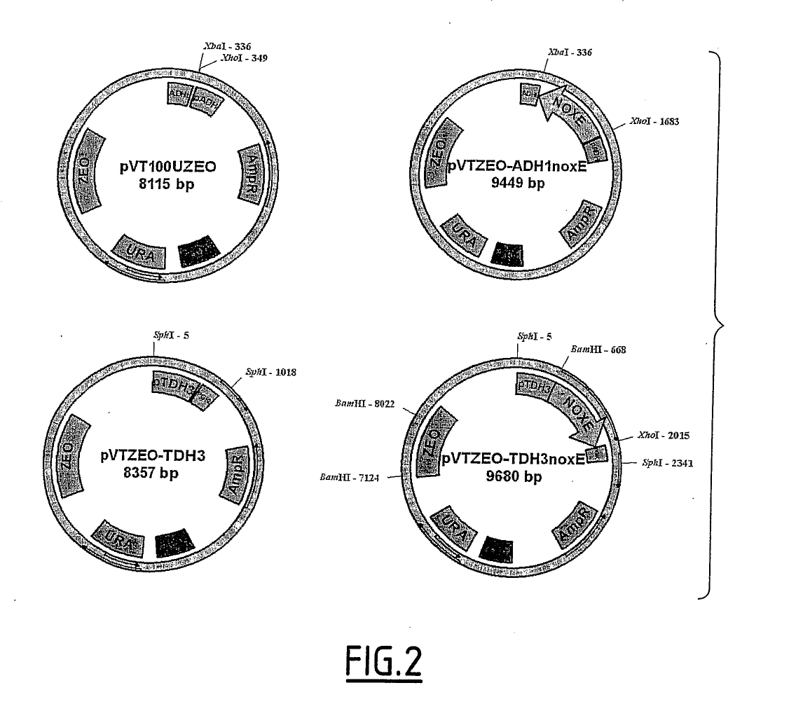

The expression plasmid pVT100-U ZEO was used (FIG. 2). That plasmid is derived from the plasmid pVT100-U described by Vernet et al. (1987), which contains the origin of replication of yeast 2μ, the selection marker URA3 and the strong regulatory elements ADH1 (alcohol dehydrogenase I promoter and terminator), as well as the bacterial elements (origin of replication and ampicillin resistance gene) into which the gene Tn5 ble conferring phleomycin resistance has been inserted, as described by Remize et al. (1999).

In order to express the oxidase, the vector pVTZEO-ADH1noxE was constructed by inserting the noxE gene (Hoefnagel et al., 2002) into the vector pVT100U-ZEO. To that end, the noxE gene was amplified by PCR as described in 5, starting from the total DNA isolated from L. lactis MG1363, using the primers 5′-CGGCGCTCGAGATGAAAATCGTAGTTATCGGT-3′ (SEQ ID No.7) and 5′-CGGCGTCTAGATTATTTGGCATTCAAAGCTGC-3′ (SEQ ID No.8) into which the XhoI and XbaI sites (underlined) have been introduced.

50 ng of fragment amplified and digested by XhoI and XbaI were ligated to 100 ng of plasmid pVT100UZEO digested by XhoI and XbaI and dephosphorylated as described in 6. After transformation of bacteria and selection of the recombinant clones as described in 6, a number of recombinant clones were obtained. The map of the resulting recombinant plasmid, called pVTZEO-ADH1noxE, is shown in FIG. 2.

2. Introduction of the NADH Oxidase on the Multicopy Plasmid pVT100-U ZEO Under the Control of the TDH3 Promoter

The vector pVTZEO-TDH3noxE was also constructed from the plasmid pVT100U-ZEO, by replacing the expression cassette ADH1p-ADH1t by a cassette constituted by the noxE gene under the control of the promoter of the yeast gene TDH3 coding for glyceraldehyde 3-phosphate dehydrogenase and of the terminator of the PGK gene coding for phosphoglycerate kinase. The TDH3 promoter has been described as a very strong and constitutive promoter (Mumberg et al., 1995).

The plasmid pVTZEO-TDH3noxE was obtained by using the intermediate plasmid pFL-TDH3noxE, which was obtained by cloning the promoter region of TDH3 into the yeast/coli shuttle vector pFL60 described by Minet et al. (1992). The promoter region of TDH3 was amplified from the genomic DNA of the yeast strain S. cerevisiae S288C, with the aid of the oligonucleotides 5′-CGGAGCTCCAGTTCGAGTTTATCATTATC-3′ (SEQ ID No.9) and 5′-CGGGATCCTCGMACTMGTTCTTGGTG-3′ (SEQ ID No.10) into which the SacI and BamHI sites (underlined) were introduced as described in 5. The coding region of the noxE gene was amplified by PCR, as described in 5, starting from the chromosomal DNA of L. lactis MG1363 with the aid of the nucleotides 5′-CGGGATCCATGMMTCGTAGTTATCGGT-3′ (SEQ ID No.11) and 5′-CGCTCGAGTTATTTGGCATTCAAAGCTGC-3′ (SEQ ID No.12) permitting the introduction of the BamHI and XhoI sites (underlined). The two PCR fragments so generated were digested and then ligated into the plasmid pFL60 digested by SacI and XhoI as described in 6. The plasmid pFL-TDH3 noxE was thus obtained. The expression cassette TDH3p-noxE-PGKt was amplified, as described in 5, starting from that plasmid using the oligonucleotides 5′-GGCGGCATGCGCTCCAGTTCGAGTTTATCA-3′ (SEQ ID No.13) and 5′-CGGCGGCATGCTTTCACACAGGAAACAGCTA-3′ (SEQ ID No.14), into each of which a site SphI (underlined) has been introduced. 100 ng of fragment amplified and digested by SphI were ligated to 100 ng of plasmid pVT100-U-ZEO digested by SphI and dephosphorylated as described in 6. After transformation of bacteria and selection of the recombinant clones as described in 6, several recombinant clones were obtained. The map of the resulting recombinant plasmid, called pVTZEO-TDH3noxE, is shown in FIG. 2.

The empty plasmid pVTZEO-TDH3 used as control was generated starting from the plasmid pFL-TDH3, which corresponds to the plasmid pFL60 in which the promoter PGK has been replaced by a multi-cloning site, with the aid of the double-stranded oligonucleotide (MWG) 5′-ATCCCCCGGGCTGCAGGTCGACC-3′ (SEQ ID No.15), then in which the promoter TDH3 has been cloned at the site SacI and BamHI as explained above. The map of the resulting recombinant plasmid, called pVTZEO-TDH3, is shown in FIG. 2.

3. Preparation of the Expression Cassette TDH3p-noxE-PGKt in Order to Integrate it into the Yeast Genome

A strain stabling expressing the noxE gene was constructed by integrating the cassette TDH3p-noxE-PGKt at locus URA3 of the strain V5 using the “short flanking homology” (SFH) method described by Guldener et al. (1996). The expression cassette TDH3p-noxE-PGKt was amplified, as described in 5, starting from the plasmid pFL-TDH3noxE using the oligonucleotides 5′-CGGCGGATATCGCTCCAGTTCGAGTTTATCA-3′ (SEQ ID No.16) and 5′-CGGCGACTAGTTTTCACACAGGAAACAGCTA-3′ (SEQ ID No.17) into which the sites EcoRV and SpeI have been introduced. The resulting amplification fragment was ligated to the plasmid pUG6 (Guldener et al., 1996) digested by EcoRV and SpeI and dephosphorylated as described in 6. The plasmid pUG6noxE was obtained.

In order to transform the strain V5 (see 4), a PCR fragment carrying the modules loxP-kanMX4-loxP and TDH3p noxE-PGKt was amplified, as described in 5, starting from the plasmid pUG6noxE, with the aid of the oligonucleotides 5′-TGATTCGGTAATCTCCGAGCAGAAGGAAGAACGAAGGAAGGCAGGTCGACMCCCTTAAT-3′ (SEQ ID No.18), which possesses 20 nucleotides complementary to pUG6 and an extension of 40 nucleotides (underlined) corresponding to the region −157 to −117 upstream of the ATG of URA3, and 5′-TGAGTTTAGTATACATGCATTTACTTATAATACAGTTTTTTTCACACAGGAAACAGCTA-3′ (SEQ ID No.19), which possesses 20 nucleotides complementary to the PGK terminator and an extension of 39 nucleotides (underlined) corresponding to the region+843 to +804 downstream of the stop codon of URA3.

4. Transformation of the Yeast

The yeast strain Saccharomyces cerevisiae ScV5M (called V5) was transformed by the vectors pVTZEO-ADH1noxE, pVTZEO-TDH3noxE and by the vectors pVT100UZEO and pVTZEO-TDH3 (controls). The resulting strains are listed in Table 1.

TABLE 1
List of plasmids and strains used.

| | | Genetic | |
|---|---|---|---|
| Strain | Genotype | modification | Source |
| V5 | MATa ura3 | | INRA UMR |
| | | | SPO |
| V5noxE | MATa ura3 | Integration URA3 | This work |
| | TDH3p-noxE-PGKt | | |
| V5pVTZEO- | MATa ura3 | Plasmid | This work |
| TDH3noxE | TDH3p-noxE-PGKt | 2μ, URA3, Ap^{R}, | |
| | | G418^{R}, ZEO^{R} | |
| V5pVTZEO- | MATa ura3 | Plasmid | This work |
| TDH3 | TDH3p-PGKt | 2μ, URA3, Ap^{R}, | |
| | | G418^{R}, ZEO^{R} | |
| V5pVTZEO- | MATa ura3 | Plasmid | This work |
| ADH1noxE | ADH1p-noxE- | 2μ, URA3, Ap^{R}, | |
| | ADH1t | G418^{R}, ZEO^{R} | |
| V5pV100U-ZEO | MATa ura3 | Plasmid | This work |
| | ADH1p-ADH1t | 2μ, URA3, Ap^{R}, | |
| | | G418^{R}, ZEO^{R} | |

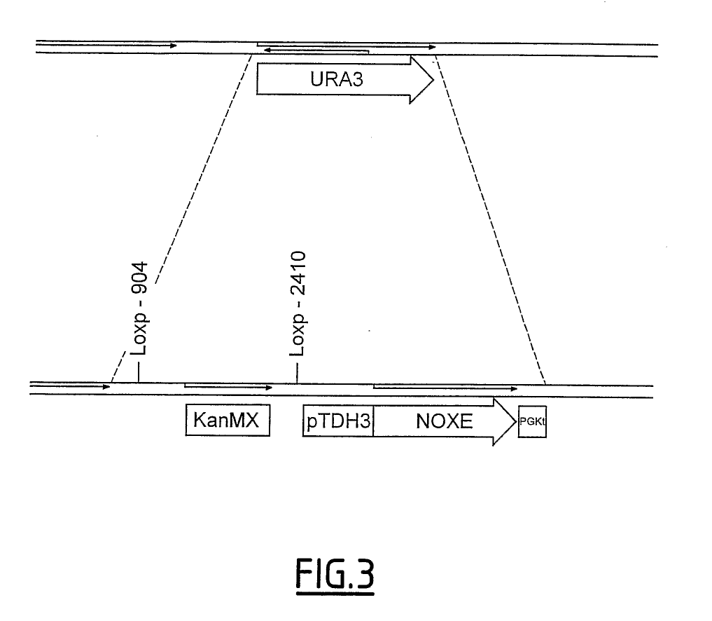

In order to obtain a strain that stably expresses the oxidase, strain V5 was transformed by 1.4 μg of integration fragment prepared as described in 3. Integration at the locus URA3 was verified by PCR starting from the genomic DNA of the resulting G418R transformants, with the aid of oligonucleotides situated upstream and downstream of the integration site. A strain called V5noxE possessing the fragment integrated at the locus URA3 was obtained (FIG. 3).

Strain SCV5M was deposited on 18 Jun. 1992 at the Collection Nationale de Cultures de Microorganismes, held by the Institut Pasteur, under number I-1222. It is a haploid S. cerevisiae strain, MATa, ura3, derived from an enological strain.

The transformation method used is the lithium acetate method described by Schiestl and Gietz (1989). The selective medium used to select the strains transformed by the plasmids is YNB (0.67% yeast nitrogen base, 2% glucose). The absence of uracil allows a selection pressure for the plasmids to be maintained.

The clones that have integrated the integration fragment carrying the modules kanMX and NADH oxidase were selected on YEPD rich medium (1% bacto yeast extract, 2% bactopeptone, 2% glucose) supplemented with 200 μg/ml of geneticin G418 (Gibco, England).

5. Amplification by PCR

50 ng of plasmid or 100 ng of genomic DNA are mixed with 500 nM of oligonucleotides, 5 μl of 10× Mg²⁺-free DyNazyme EXT buffer (FINNZYMES, Finland), 1.5 mM of MgCl₂, 200 μM of dNTPs, 1 unit of DyNAzyme EXT (FINNZYMES, Finland) in a total volume of 50 μl.

The amplification conditions are as follows: 2 minutes at 94° C., 30 cycles of 30 seconds at 94° C., 30 seconds at 50° C., 2 minutes at 72° C., then 7 minutes at 72° C. on amplification Perkin-Elmer Cetus model 9600.

6. Bacteria Cloning and Transformation Techniques

The digestion of the DNA by restriction enzymes is carried out as described by the supplier (Promega Corporation, USA). After digestion, the plasmids are dephosphorylated with 10 units of bacterial alkaline phosphatase (Qbiogene, USA) according to the protocol described by the supplier. The dephosphorylation reaction is stopped by extraction with phenol/chloroform (Sambrook et al., 1989). From 50 ng to 100 ng of amplified and digested DNA are ligated overnight at 16° C. to 100 ng of digested and dephosphorylated plasmid in a final reaction mixture of 10 μl, in the presence of 5 units of T4 DNA ligase (Biolabs, USA). One microliter of ligation mixture is used to transform the competent E.coli DH5α bacteria (Library Efficiency DH5α competent cells, Invitrogen, USA) according to the protocol described by the supplier. The resulting colonies are selected on LB dishes (1% bactotryptone, 0.5% bacto yeast extract, 1% NaCl) plus ampicillin (100 μg/ml). The plasmid DNA of the resulting clones is then extracted using the QlAprep Miniprep kit (Quiagen, USA) and analyzed by enzymatic digestion.

### Example 2

### Consequences of the Expression of the NADH Oxidase in Saccharomyces cerevisiae During Fermentation on MS Medium, Under Constant Oxygen Supply Conditions

1. Experimental Procedures

a—Culture and Microoxygenation Conditions

The fermentations were carried out in 2-liter reactors (SGI, France) with a reaction volume of 1 liter. MS medium was used for the preculture and the culture. It is a synthetic medium which simulates a standard grape must (Bely et al., 1990). MS medium comprises from 18 to 20% glucose, 6 g/l of malic acid, 6 g/l of citric acid, 460 mg/l of nitrogen, in the form of NH₄Cl (120 mg/l) and of amino acids (340 mg/l). The medium is supplemented with methionine (115 mg/l) and, if necessary, uracil (50 mg/l). The pH of MS medium is 3.3. Anaerobiosis factors, ergosterol (7.5 mg/l), oleic acid (2.5 mg/l) and Tween 80 (0.21 g/l) are added. The precultures are carried out in 250 ml Erlenmeyer flasks containing 50 ml of medium at 28° C. with stirring (150 rpm) for 30 hours. The reactors are inoculated starting from those precultures, to a cell density of 1.10⁶ cells/ml, and maintained at a constant temperature of 28° C. with permanent stirring (500 rpm). The microoxygenation conditions are obtained by aerating the reactor with air at a constant flow rate. Measurement of the dissolved oxygen is carried out by means of INGOLD Clark electrodes. The transfer coefficient (Kₗa) is measured according to the dynamic method (Dursun et al., 1999). The oxygen solubility (C*) in the must is determined according to Sablayrolles and Barr (1986). The maximum oxygen transfer rate (OTR max) is equal to kₗa×C* and the oxygen consumption rate (OUR) is calculated as follows: OUR=kₗa×(C*−C), where C is the dissolved oxygen concentration in the medium. The oxygen consumption is obtained by integrating the curve obtained when calculating the OUR. The outlet gas passes through a refrigerated condenser in order to avoid evaporation of the volatile compounds.

The culture samples were collected by means of a syringe. The fermentation data are expressed as a function of time or of the progress of the reaction, 1-S/S0 where S=glucose concentration and S0=initial glucose concentration.

b—Analytical Methods

The growth is monitored by measuring the optical density at 600 nm and by counting the number of cells on an apparatus of the Coulter Counter type (ZBI) on a sample of an aliquot fraction of culture medium.

The metabolites are titrated in the supernatant, after centrifugation of the samples taken at 13,000 rpm for 5 minutes. The glucose, glycerol, ethanol, pyruvate, succinate, acetate, α-ketoglutarate and 2-hydroxyglutarate concentration is determined by high-pressure liquid chromatograpy (HPLC) using a column of the HPX-87H type (Bio-Rad). The acetaldehyde concentration is determined by the enzymatic method described by Lundquist (1974). The acetoin and 2,3-butanediol concentration was determined by gas-phase chromatography as described above (Michnick et al., 1997).

c—Cell Extracts and NADH Oxidase Activity

The yeast cell extracts were prepared starting from 1×10⁹ cells collected in the reactor. After centrifugation for 5 minutes at 3000 rpm, the cells are washed with a KH₂PO₄ 100 mM buffer, pH 7 and then with a KH₂PO₄ 10 mM buffer, pH 7. 100 mg of yeast (wet weight) are then ground by addition of 1 g of glass beads (Ø=0.5 mm) and 0.5 ml of KH₂PO₄ 50 mM buffer, pH 7 comprising 1 mM DTT and 2 mM MgCl₂. The tube is agitated for 1 minute under a vortex and placed in ice for 1 minute. The operation is repeated 5 times. After centrifugation for 1 minute at 13,000 rpm, the supernatant is recovered and constitutes the cell extract.

The enzymatic activities are determined extemporaneously. The specific NADH oxidase activity in the cell extracts is measured using a spectrophotometer at 25° C. in a total volume of 1 ml comprising 50 mM potassium phosphate buffer (pH 7), 0.3 mM NADH and 0.3 mM EDTA. The reaction is initiated by addition of from 5 to 50 μl of cell extract and monitored by the diminution in absorbance at 340 nm. The protein concentration is determined using the BC Assay kit (Uptima, Interchim).

d—Extraction and Measurement of the Intracellular NADH/NAD Contents

The metabolites are extracted as described by Gonzalez et al. (1997). Five milliliters of cell culture are added to 26 ml of an iced solution comprising 60% (vol/vol) methanol and 175 mM HEPES (pH 7.5). The mixture is centrifuged at 5000 g for 30 seconds at −10° C. The intracellular metabolites are extracted with 5 ml of a boiling solution of absolute ethanol/1M HEPES (pH 7.5)/H₂O (750/70/180 vol/vol/vol) and incubated for 5 minutes at 80° C. The extracts are placed in ice for 5 minutes. After addition of 2 ml of absolute ethanol, the extracts are vacuum-dried for 4 minutes at 70° C. in a rotary evaporator (model Laborota 4000; Heidolph Instruments LLC, Cinnaminson, N.J.). The residue is resuspended in a final volume of from 1 to 2 ml of distilled water and stored at −80° C. for subsequent use. The volume of extract is measured by weighing.

The cofactors concentrations are determined from enzymatic reactions coupled to NAD(H)— as described below.

The amount of NADH produced during the reaction is determined by fluorescence spectrophotometry (excitation wavelength 340 nm; emisson wavelength 460 nm) using a Perkin Elmer LS 50B fluorescence spectrophotometer. The enzymatic reactions are carried out at 30° C. in a total volume of 2 ml of reaction buffer comprising 4.25 mM Tris-NH₄Cl (pH 7.0), 25 μM dihydroxyacetone phosphate and 125 μM α-ketoglutarate, as described by Klingenberg (1974). Aliquot fractions of from 5 to 100 μl of samples are added to the reaction buffer. A base line is obtained. One microliter of glycerol-3-phosphate dehydrogenase (170 U·ml⁻¹; Roche) then 1 μl of NADPH-dependent glutamate dehydrogenase (240 U·ml⁻¹; Roche) are added in succession. Each addition is carried out after a stable signal has been obtained.

The NAD concentration is determined as described above (Bergmeyer, 1955). The reaction buffer comprises 1.8 ml of a mixture of 0.2 M glycine and 0.4 M hydrazine hydrate (pH 9), 85 mM ethanol and from 5 to 200 μl of extract in a total volume of 2.01 ml. After the base line has been obtained, 1 μl of alcohol dehydrogenase (882 U·ml⁻¹; Roche) is added.

The cofactors concentrations in the samples are calculated by an external calibration method, allowing the response coefficient of each cofactor to be determined. The measurements are carried out in triplicate.

2. Results

Fermentations in batch mode, under microoxygenation conditions (see 5a), were carried out in order to analyze the impact of the expression of the oxidase on the growth, the sugar degradation, the production of metabolites and the intracellular concentration of cofactors NAD, NADH.

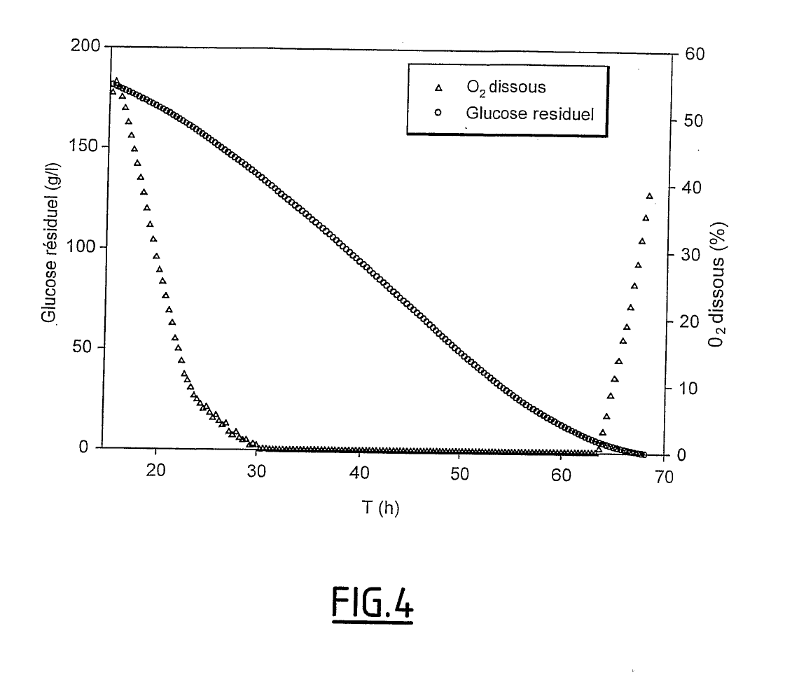

The fermentations were carried out with strains expressing NADH oxidase V5noxE, V5pVTZEO-TDH3noxE, V5pVTZEO-ADH1noxE and the control strains V5, V5pVTZEO-TDH3 and V5pVTZEO-ADH1. The flow rate of air used in this experiment is kept constant throughout the fermentation at 17 ml/minute, which corresponds to an oxygen transfer rate of 10 mg/l/h. Under those conditions, the totality of the oxygen is consumed by the control strain V5 (FIG. 4).

The specific NADH oxidase activity was measured in the different strains at 2 stages of the fermentation (Table 2) in exponential phase medium (17 h culture) and in stationary phase (40 h).

TABLE 2
Specific NADH oxidase activity of the strains V5, V5 pVT100-UZEO,
V5noxE, V5 pVTZEO-TDH3noxE, V5 pVT ZEO-ADH1noxE
on MS medium with a constant oxygen supply of 10 mg/l/h.
Specific NADH oxidase activity in U/mg of protein

| Strain | Exponential growth phase | Stationary phase |
|---|---|---|
| V5 | nd | nd |
| V5noxE | 0.36 ± 0.04 | 0.14 ± 0.01 |
| V5 pVTZEO-TDH3noxE | 1.48 ± 0.13 | 0.51 ± 0.02 |
| V5 pVTZEO-TDH3 | nd | nd |
| V5 pVTZEO-ADH1noxE | 0.28 ± 0.03 | 0.59 ± 0.07 |
| V5 pVT100-UZEO | nd | nd |

nd: not detected

Under enological conditions, the growth phase is short. The exhaustion of assimilable nitrogen from the medium causes rapid entry into the stationary phase (after about 30 hours), while approximately 30% of the initial sugars are consumed. The stationary phase therefore represents an important phase during which the majority of the sugars (about 70%) is degraded.

As expected, no NADH oxidase activity is detected in the cell extracts of the control strain and of the strains transformed by the empty plasmids, while a significant activity is measured in the strains expressing the noxE gene, which indicates that the enzyme coded for by the bacterial noxE gene is expressed well in S. cerevisiae. The maximum activity obtained (1.48 U/mg of protein) is about 7 times greater than that measured in a cell extract of L. lactis (Lopez de Felipe and Hugenholtz, 2001).

NADH oxidase is expressed throughout the entire fermentation time, with a level of specific activity that is about 3 times higher in the growth phase compared with the stationary phase when the noxE gene is placed under the control of the TDH3 promoter. The reverse is obtained with the ADH1 promoter, the level of specific activity is twice higher in the stationary phase than in the growth phase. The level of activity varies considerably depending on the promoter used and the number of copies of the gene. With the TDH3 promoter it is possible to obtain an activity that is 5 times greater than that obtained with the ADH1 promoter in the growth phase, while the levels of specific activity are similar for both constructs in the stationary phase. When noxE is placed under the control of TDH3, the activity obtained in multicopy is about 3 times higher than that obtained in the strain that has integrated that cassette in a single copy.

The impact of the expression of the oxidase was analyzed by monitoring the growth, the glucose degradation, and the formation of ethanol, glycerol, acetate, acetaldehyde, pyruvate, α-ketoglutarate, 2-hydroxyglutarate, acetoin, butanediol and biomass during the fermentation. Table 3 shows the yields of biomass and products obtained after stopping the fermentation.

TABLE 3
Production yield of the principal fermentary metabolites, biomass, carbon balance and degree of reduction of the strains V5,
V5 pVT100-UZEO, V5noxE, V5 pVTZEO-TDH3noxE, V5 pVT ZEO-ADH1noxE on MS medium with a constant oxygen supply of 10 mg/l/h

| | Progress | Yield (g of metabolite produced/g of glucose consumed)^{b,c} | | | | | | | | | | | Carbon |
|---|---|---|---|---|---|---|---|---|---|---|---|---|---|
| | of the | | | Glyc- | | Acetal- | | α-keto- | 2-Hydroxy- | | | Bio- | balance |
| Strains | reaction | Ethanol | CO₂^{a} | erol | Acetate | dehyde | Pyruvate | glutarate | glutarate | Acetoin | Butanediol | mass | (%) |
| V5 | 0.5 | 0.464 | 0.232 | 0.042 | 0.010 | 0.004 | 0.0027 | 0.025 | 0.0025 | 0.025 | 0.010 | 0.064 | 104.5 |
| | | (0.009) | (0.005) | 0.001) | (0.001) | (0.001) | (0.0001) | (0.002) | (0.0001) | (0.010) | (0.002) | | |
| | 1 | 0.460 | 0.230 | 0.028 | 0.013 | 0.003 | 0.0007 | 0.014 | 0.0015 | 0.026 | 0.012 | 0.031 | |
| | | (0.000) | (0.000) | (0.000) | (0.001) | (0.000) | (0.0002) | (0.001) | (0.0001) | (0.009) | (0.001) | | |
| V5 pVT100- | 0.5 | 0.463 | 0.232 | 0.038 | 0.009 | 0.005 | 0.0024 | 0.002 | 0.0017 | ND | ND | 0.059 | 96.8 |
| UZEO | | (0.004) | (0.002) | (0.012) | (0.000) | (0.001) | (0.0005) | (0.001) | (0.0002) | | | | |
| | 1 | 0.457 | 0.229 | 0.025 | 0.012 | 0.002 | 0.0006 | 0.001 | 0.0016 | ND | ND | 0.031 | |
| | | (0.003) | (0.002) | (0.006) | (0.000) | (0.000) | (0.0001) | (0.000) | (0.0001) | | | | |
| V5pVTZEO- | 0.5 | 0.459 | 0.230 | 0.035 | 0.010 | 0.004 | 0.0016 | 0.003 | 0.0038 | ND | ND | 0.059 | 97.6 |
| TDH3 | | (0.003) | (0.002) | (0.002) | (0.000) | (0.000) | (0.0001) | (0.000) | (0.0007) | | | | |
| | 1 | 0.472 | 0.236 | 0.021 | 0.012 | 0.002 | 0.0008 | 0.002 | 0.0024 | ND | ND | 0.025 | |
| | | (0.004) | (0.002) | (0.000) | (0.000) | (0.000) | (0.0000) | (0.000) | (0.0003) | | | | |
| V5noxE | 0.5 | 0.398 | 0.199 | 0.032 | 0.034 | 0.013 | 0.0041 | 0.003 | 0.0008 | 0.063 | 0.011 | 0.025 | 101 |
| | | (0.002) | (0.001) | (0.001) | (0.003) | (0.002) | (0.0005) | (0.000) | (0.0000) | (0.005) | (0.000) | | |
| V5pVTZEO- | 0.5 | 0.397 | 0.199 | 0.027 | 0.027 | 0.011 | 0.0037 | 0.002 | 0.0022 | ND | ND | 0.023 | 89.5 |
| ADH1noxE | | (0.002) | (0.001) | (0.006) | (0.001) | (0.004) | (0.0005) | (0.001) | (0.0001) | | | | |
| V5pVTZEO- | 0.5 | 0.393 | 0.197 | 0.030 | 0.032 | 0.011 | 0.0044 | 0.005 | 0.0013 | ND | ND | 0.028 | 89.7 |
| TDH3noxE | | (0.001) | (0.000) | (0.000) | (0.002) | (0.002) | (0.0002) | (0.000) | (0.0001) | | | | |

^{a}CO₂ estimated from ethanol production
^{b}The values in parentheses are the standard deviations over 2 experiments
^{c}ND: not determined

Under the microoxygenation conditions used, the 3 strains expressing the oxidase consume only half (i.e. about 100 g/l) of the sugars present, unlike the control strains, which complete fermentation. The effects of the oxidase on the central metabolism were therefore analyzed by comparing the yields of biomass and principal fermentary by-products at mid-fermentation (progress of the reaction 0.5). By way of information, the yields obtained after degradation of the totality of the sugars (200 g/l) are indicated for the control strains (progress of the reaction 1).

A very significant reduction (15%) in the ethanol yield is observed for the 3 strains expressing NADH oxidase as compared with the control strains cultivated under the same conditions (non-transformed strain and strain carrying an empty plasmid).

In addition, the central metabolism of the strains expressing oxidase is particularly affected. The production yield of acetaldehyde, acetate, pyruvate and acetoin is increased in response to the slowing down of the carbon flux towards the production of ethanol, while that of glycerol is reduced. Because the presence of empty plasmid (pVTZEO-ADH1 and pVTZEO-TDH3) interferes with the production of α-ketoglutarate and 2-hydroxyglutarate, only the differences observed between the strains V5 and V5noxE are taken into account, showing a considerable reduction in the production of those compounds in the strain V5noxE.

The analysis of the results (Table 3 and kinetic data not shown) does not reveal any significant difference between the three strains expressing oxidase, either at the level of growth or of the metabolic profiles, despite very different levels of activity (Table 2).

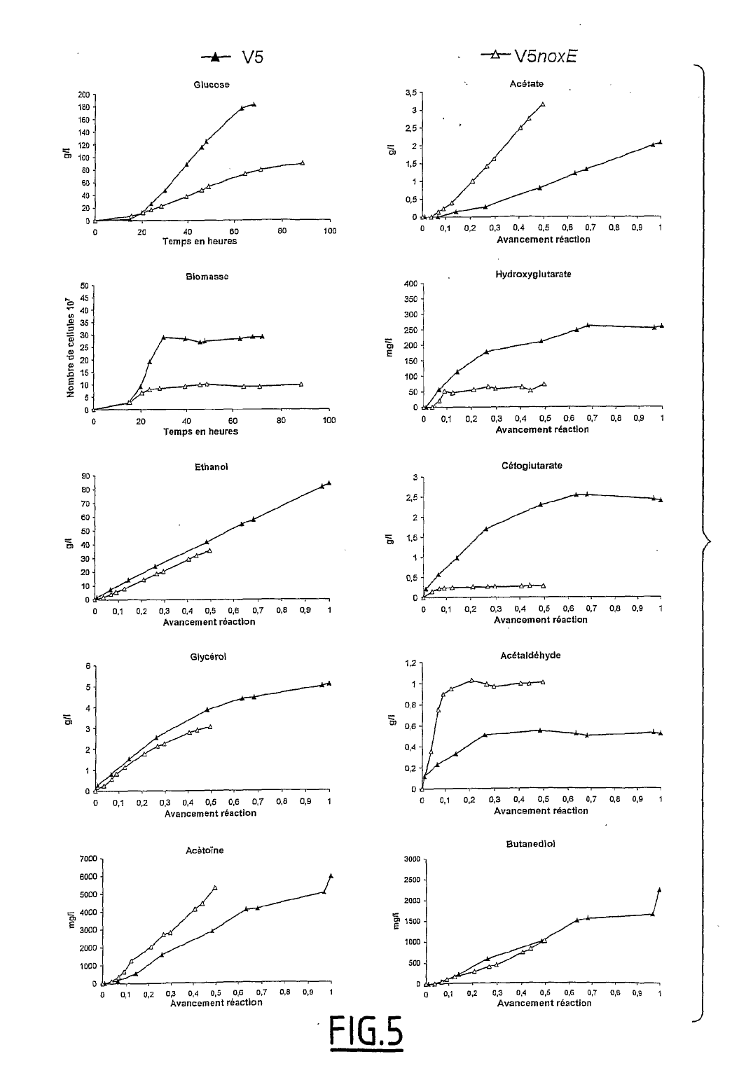

The detailed analysis of the impact of the expression of the oxidase on growth, the rate of fermentation and the metabolic profiles is shown only for the strain V5noxE, in comparison with the strain V5 (FIG. 5). The reduction in the production yield of ethanol in the strain V5noxE is visible from the start of the fermentation. That reduction would lead to a reduction of 17 g/l of ethanol, i.e. 20 of alcohol in the finished product if all the glucose was consumed. At the end of the fermentation, 100 g of glucose out of 200 g have been consumed by the strain V5noxE.

The production of glycerol is also reduced from the start of fermentation. The oxidase, by reoxidizing part of the intracellular NADH, therefore enters into competition with other yeast enzymes that use that cofactor. In particular, the two NADH-dependent reactions catalyzed by alcohol dehydrogenase (ADH) and glycerol 3-P dehydrogenase (GPDH) are limited owing to the reduced availability of NADH. These results show a very efficient use of that cofactor by NADH oxidase, in agreement with a Km NADH of 4.1 μM determined on the purified enzyme of L. lactis (Lopez de Felipe and Hugenholtz, 2001). The affinity of NADH oxidase for NADH is higher than that of yeast ADH (110 μM) and GPDH (23 μM) (Teusink et al., 2000).

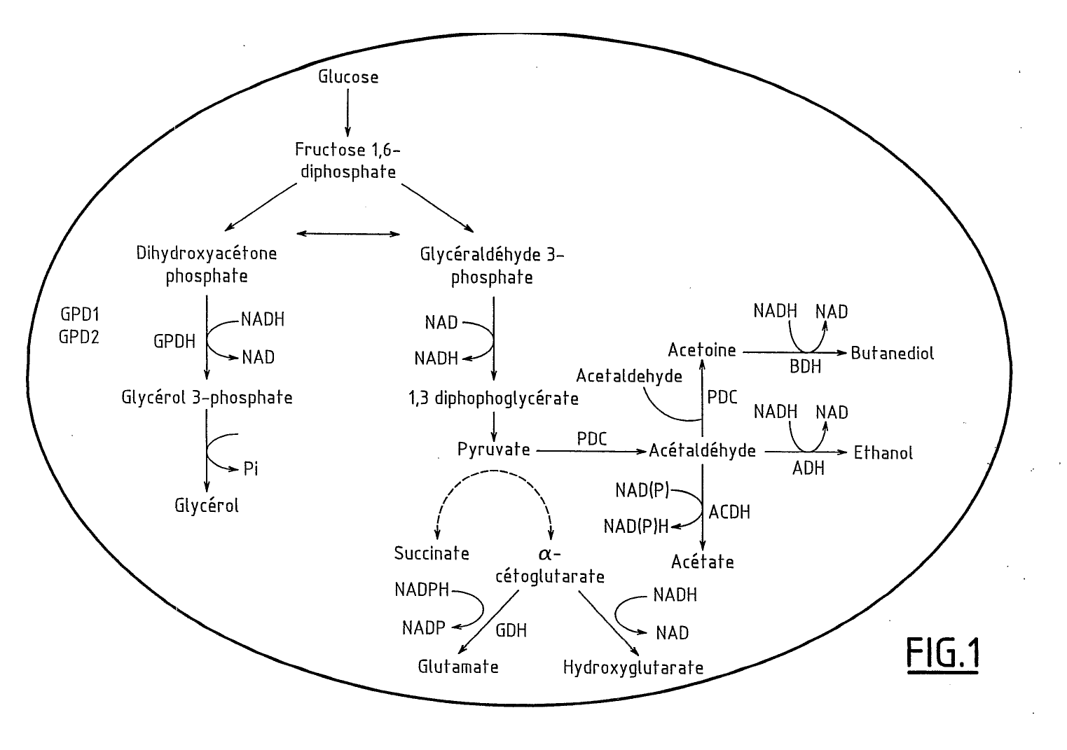

The first consequence of limiting the flow of carbon towards the ethanol synthesis is an increase in the production of acetaldehyde and acetate in the transformants. The synthesis of acetate principally generates NADPH, via the acetaldehyde dehydrogenases Ald6p and Ald5p located in the cytoplasm and in the mitochondrium, respectively (Saint-Prix et al., 2004). The strain V5noxE also exhibits a considerable reduction in the production of α-ketoglutarate. This effect may be due to the surplus of NADPH linked to the increase in acetate synthesis. In fact, the synthesis of glutamate from α-ketoglutarate via glutamate dehydrogenase is a route which consumes a large amount of NADPH (Nissen et al., 1997) (FIG. 1). The fact that a mutant V5ald6 exhibits an increase in the production of α-ketoglutarate in response to a NADPH deficit (Saint-Prix et al., 2004) goes in the direction of this hypothesis.

Hydroxyglutarate is a reduced form of α-ketoglutarate (FIG. 1) and could act as a NADH redox valve (Albers et al., 1998). The reduction in its production in the strain V5noxE might therefore come from a reduced availability of substrate (α-ketoglutarate) and/or from a reduced availability of NADH due to the competition with the NADH oxidase. Note that the dehydrogenase involved, which is NADH-dependent, is not identified in S. cerevisiae.

The increase in acetate may be linked to the accumulation of its precursor, acetaldehyde, the production of which is increased in a drastic and very early manner. It is interesting to note an early stopping (about 20 h) of the growth of the strain V5noxE, while the concentration of acetaldehyde in the medium at that stage reaches 1.1 g/l instead of 0.2 g/l for the control strain. The number of cells achieved by V5noxE is three times smaller than that of the wild-type strain. Acetaldehyde is a compound which is toxic to yeast. It has a negative effect on the formation of biomass (Aranda and del Olmo, 2004; Liu and Pilone, 2000) and, at a high concentration, on the fermentation rate (Roustan and Sablayrolles, 2002). Its strong polarity induces hydric stress in yeast (Hallsworth, 1998;. Liu and Pilone, 2000). The pronounced accumulation of acetaldehyde might therefore be responsible for the negative effects of the oxidase on the growth and fermentability of sugars. That toxicity might be disadvantageous in the presence of a strong expression of the oxidase and might explain that the same effect is obtained when the noxE gene is expressed in multiple copies or integrated.

In yeast, acetaldehyde can be metabolized to acetoin and 2,3-butanediol (FIG. 1), which compounds are not toxic to yeasts. Acetoin is produced by the condensation of 2 molecules of acetaldehyde by pyruvate decarboxylase (PDC) and then reduced to 2,3-butanediol by butanediol dehydrogenase (BDH). That reduction is NADH-dependent (Gonzalez et al., 2000). Titration of those compounds in the strains V5 and V5noxE shows that the level of acetoin is almost doubled in the strain V5noxE, while the production of butanediol remains similar for the 2 strains. These results show that NADH oxidase also enters into competition with BDH whose Km is 55 μM for NADH. Owing to the slowing down of that reaction, the accumulated acetaldehyde cannot be resorbed and affects both the growth and the fermentary activity. In support of this hypothesis, it has been observed that the addition of 900 mg/l of acetaldehyde affects the growth of the strain V5 temporarily, the conversion of that compound into acetoin then into 2,3-butanediol permitting a rapid resumption of growth.

In order to evaluate the impact of the oxidase on the intracellular oxidoreduction equilibrium, we measured the intracellular concentration of reduced and oxidized cofactors NADH and NAD at two different stages of the alcoholic fermentation under controlled microoxygenation conditions with a constant transfer rate of 10 mg/l/h. The results, given in Table 4, show a very marked reduction in the intracellular concentration of NADH, which falls by 80% as compared with the control strain. These data show that NADH oxidase drastically affects the NADH/NAD pool and that yeast, under those conditions, is not capable of reequilibrating its oxidoreduction balance. The NADH/NAD ratio is in fact very reduced both in the growth phase and in the stationary phase, as compared with the control strain.

TABLE 4
Intracellular concentrations of NAD and NADH of the strains V5 and
V5noxE on MS medium with a constant oxygen supply of 10 mg/l/h.
Cofactor concentration in μmoles/g of biomass

| | Exponential growth phase | | | Stationary phase | | |
|---|---|---|---|---|---|---|
| Strain | NADH | NAD | NADH/NAD | NADH | NAD | NADH/NAD |
| V5 | 0.36 ± 0.05 | 2.70 ± 0.08 | 0.13 | 0.15 ± 0.04 | 2.83 ± 0.02 | 0.05 |
| V5noxE | 0.07 ± 0.00 | 3.93 ± 0.00 | 0.02 | 0.04 ± 0.02 | 3.75 ± 0.10 | 0.01 |

In conclusion, these data show that the expression of a NADH oxidase in S. cerevisiae reduces the intracellular NADH pool considerably, bringing about a significant deviation in the carbon fluxes, which is reflected especially in a very significant reduction in the production yield of ethanol, an increase in acetaldehyde and acetate and acetoin derivatives, and a reduction in glycerol, α-ketoglutarate and hydroxyglutarate formation. The rapid and considerable accumulation of acetaldehyde causes disadvantageous effects at the level of the fermentation and growth. In fact, that compound cannot be eliminated by the cell, owing to a reduction in the effectiveness of the detoxication system constituted by BDH, due to a deficit of NADH. In order to limit those undesirable effects, the inventors studied the impact of a.reduction in the oxygen supply (Example 3) and then of an oxygen supply limited to the stationary phase (Example 4).

### Example 3

### Expression of the NADH Oxidase During Fermentation with Different Oxygen Supplies

1. Culture and Microoxygenation Conditions

The conditions used are as described in Example 2, paragraph a.

2. Analytical Methods

The analytical methods are as described in Example 2, paragraph b.

3. Results

Fermentations in batch mode were carried out with different microoxygenation conditions in order to determine the minimum oxygen supply necessary for a deviation in the carbon flux. Indeed, while the wild-type strain consumes the totality of the oxygen supplied at a rate of 10 mg/l/h (FIG. 4), the strain V5noxE does not consume all the oxygen supplied, probably owing to the reduction in biomass.

The five oxygenation conditions tested on the strain V5noxE correspond to maximum oxygen transfer rates of 2, 4, 6, 7 and 10 mg/l/h. For each condition, the production of the different metabolites and the consumption of the oxygen were monitored throughout the fermentation. Table 5 shows the effects obtained on the production yield of ethanol, the accumulation of acetaldehyde, glucose degradation, growth and oxygen consumption.

TABLE 5
Dissolved O₂, ethanol yield, acetaldehyde concentration,
glucose consumption and final biomass for the strain V5 at
10 mg/l/h of transferred O₂ and for the strain V5noxE
at different transfer rates (OTR).

| | | | | Glucose | |
|---|---|---|---|---|---|
| | | Residual | Ethanol | consumed | Final |
| | OTR max | O₂^{a} | yield^{b} | (% initial | biomass |
| Strain | (mg/l/h) | (%) | (g/g) | glucose) | (×10⁷¢) |
| V5 | 10 | 0 | 0.460 | 100 | 27 |
| V5noxE | 10 | 38 | 0.397 | 48 | 9 |
| | 7 | 27 | 0.396 | 46.5 | 9 |
| | 6 | 24 | 0.379 | 45 | 9 |
| | 4 | 0 | 0.469 | 97 | 10 |
| | 2 | 0 | 0.481 | 100 | 17 |

^{a}percentage of air saturation of the medium, the probe indicates 100% when the oxygen concentration in the medium (MS 20% glucose) is 6.4 mg/l.
^{b}g of ethanol produced per g of glucose consumed.

The reduction in the ethanol yield observed for the strain V5noxE at 10 mg/l/h of transferred O₂, as compared with the strain V5, is observed with the same intensity if the transfer rate is reduced to 7 and 6 mg/l/h. At those three supply rates, the O₂ is not limiting for the strain V5noxE because the minimum quantities of dissolved O₂ measured in the medium are 38, 27 and 24%, respectively. On the other hand, under supply conditions of 4 and 2 mg/l/h of transferred O₂, the oxygen becomes limiting for the strain V5noxE. Under those conditions, the production yield of ethanol is similar to that obtained for the strain V5 with the supply of O₂ at 10 mg/l/h.

The reduction in the production yield of ethanol is accompanied by an accumulation of acetaldehyde, correlated with a reduction in the biomass of about 60% and incomplete consumption (about half) of the substrate. The lack of growth is also observed in the two cases where the O₂ is not limiting, although it is less marked for 2 mg/l/h of transferred O₂.

Under all the oxygenation conditions tested, the production of glycerol remains lower than that obtained for the wild-type strain cultivated at 10 mg/l/h of O₂, which indicates that the supply of O₂ is sufficient to permit functioning of the oxidase.

These data lead to the conclusion that the NADH oxidase is functional with very low O₂ transfer rates, but that the oxygen must preferably be supplied under non-limiting conditions if a significant reduction in the production yield of ethanol is to be observed.

### Example 4

### Expression of the NADH Oxidase During Fermentation with Oxygen Supply Limited to the Stationary Phase (Separation of the Growth Phase from the Expression Phase)

1. Culture and Microoxygenation Conditions

The conditions are as described in Example 2, paragraph a.

2. Analytical Methods

The analytical methods are as described in Example 2, paragraph b.

3. Results

In order to limit the secondary effects on the growth and fermentability of the sugars linked to the expression of the oxidase, the activity phase of the NADH oxidase was separated from the growth phase. To that end, fermentations in batch mode were carried out under anaerobic conditions up to 28 hours' fermentation (end of the growth phase), then under controlled microoxygenation conditions from the start of the stationary phase and then throughout that phase.

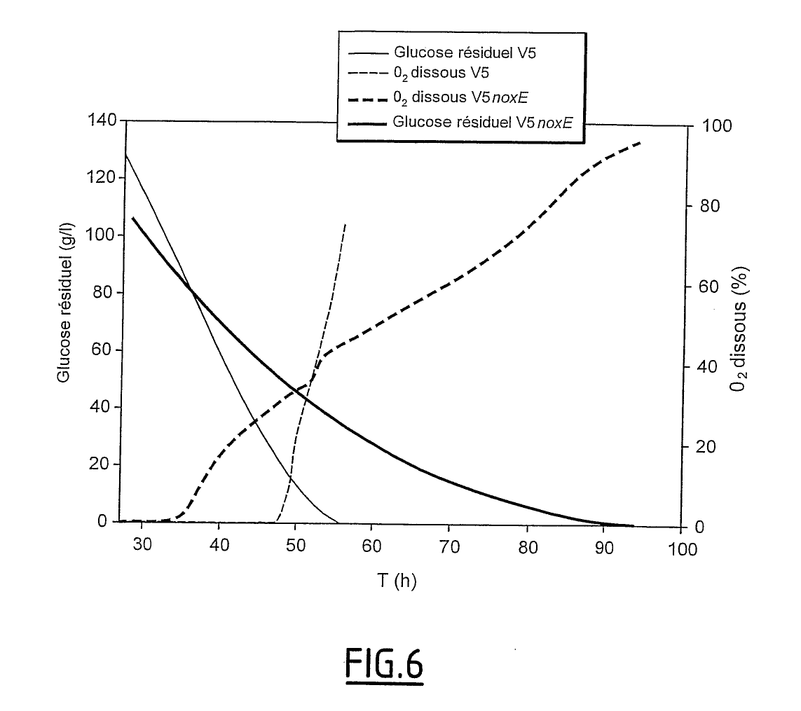

The fermentations were carried out using the NADH-oxidase-expressing strain V5noxE and the control strain V5. An air flow of 17 ml/minute, corresponding to a maximum oxygen transfer rate of 10 mg/l/h, is imposed starting at 28 hours and is maintained constant until the end of the fermentation. Under those conditions, the totality of the oxygen is consumed by the control strain V5, while the oxygen remains for the most part non-limiting for the strain V5noxE (FIG. 6).

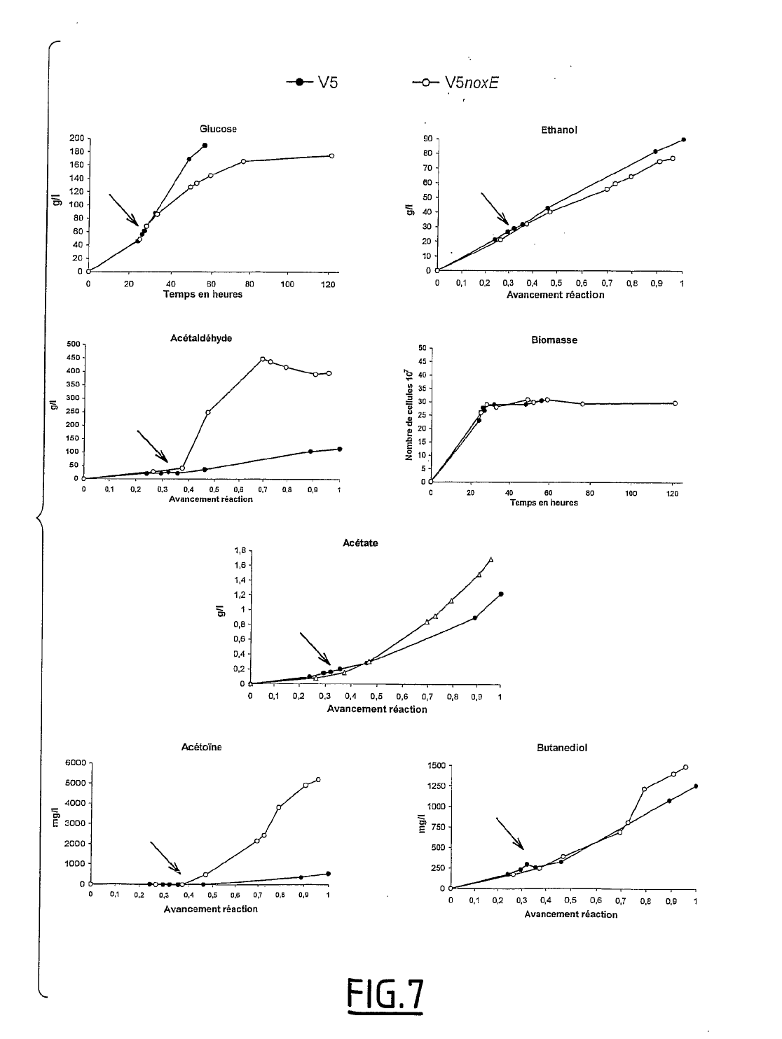

The effects obtained on growth, on glucose degradation, and on ethanol, acetate, acetaldehyde, acetoin and butanediol formation are shown in FIG. 7.

An oxygen supply limited to the stationary phase, during which phase 70% of the glucose is consumed, permits a reduction in the ethanol content of 7 g/l, i.e. 1°, as compared with the control strain cultivated under the same conditions.

The strain V5noxE, under those conditions, has identical growth with that of the control strain, an identical final biomass (30.10⁷ cells) and is capable of fermenting almost all of the glucose present (close to 200 g/l). Separating the growth phase from the phase of NADH oxidase activity therefore allows the secondary effects observed previously, on the growth and fermentability, to be avoided.

The oxygen supply unexpectedly causes an increase in the formation of acetaldehyde, which remains limited to 400 mg/l, however, which concentration does not drastically affect the fermentability. The production of acetate is increased slightly as compared with the control strain cultivated under the same conditions. The carbon flux is also reoriented towards the formation of acetoin, while the production of 2,3-butanediol remains similar between the 2 strains and is not affected by the O₂ supply.

The results obtained were compared with those obtained during a standard enological fermentation, carried out under the same conditions, but in the absence of oxygen supply (conditions of strong anaerobiosis) (Table 6).

TABLE 6
Final concentration of the main fermentary metabolites and production of
the biomass of strains V5 and V5noxE on MS under conditions of supply
of 10 mg/l/h of oxygen in stationary phase and under anaerobiosis.
Final concentrations (in g/l) on MS 20% glucose

| | Supply of 10 mg/l/h of O₂ at | | | |
|---|---|---|---|---|
| | 28 hours' culture | | Anaerobic conditions | |
| Compound | V5 | V5noxE | V5 | V5noxE |
| Glucose | 200 | 200 | 200 | 200 |
| consumed | | | | |
| Ethanol | 94.8 | 88 | 95.4 | 96.2 |
| CO₂^{a} | 47.47 | 44 | 47.8 | 48.1 |
| Glycerol | 5.6 | 5.2 | 6.2 | 6 |
| Acetate | 1.2 | 2 | 0.6 | 0.6 |
| Acetaldehyde | 0.2 | 0.4 | 0.02 | 0.02 |
| Pyruvate | 2.6 | 2 | 0.18 | 0.26 |
| α-ketoglutarate | 1.2 | 1.4 | 0.8 | 0.8 |
| Hydroxyglutarate | 0.2 | 0.2 | 0.2 | 0.2 |
| Succinate | 0.6 | 0.6 | 0.4 | 0.4 |
| Acetoin | 0.6 | 6 | 0 | 0 |
| Butanediol | 1.2 | 1.8 | 0.80 | 0.80 |
| Biomass | 6 | 6 | 4 | 4 |

^{a}CO₂ estimated from ethanol production

Under anaerobiosis, the strain V5noxE behaves like the control strain, the oxidase being inactive. The production of ethanol by the strain V5noxE under microoxygenation conditions limited to the stationary phase is reduced by 8 g/l as compared with that of the wild-type strain under anaerobiosis. Compared with the conventional process, the use of a strain expressing NADH oxidase under controlled microoxygenation conditions separated from the growth phase permits a reduction in the degree of ethanol, which in this example reaches 1° alcohol.

Under the conditions used in this example, the oxygen is limiting during the first hours of the supply, then non-limiting during the major part of the oxygenation phase (FIG. 6).

In general, those data show that limiting the supply of oxygen to one phase of the fermentation process (here the stationary phase) permits a significant reduction in the production of ethanol while remaining compatible with the physiology of yeast and its technological performances. The expression of the oxidase allows the metabolic profiles to be modified significantly. A fine modulation of the quantity and duration of the O₂ supplies during the process of fermentation of the strain expressing NADH oxidase allows not only the level of reduction of ethanol formation, but also the formation of other fermentation by-products to be optimized.

### REFERENCES

Albers, E., Gustafsson, L., Niklasson, C. and Liden, G. (1998). Distribution of 14C-labelled carbon from glucose and glutamate during anaerobic growth of Saccharomyces cerevisiae. Microbiology 144, 1683-90.

Aranda, A. and del Olmo, M. L. (2004). Exposure of Saccharomyces cerevisiae to acetaldehyde induces sulfur amino acid metabolism and polyamine transporter genes, which depend on Met4p and Haa1p transcription factors, respectively. Appl Environ Microbiol 70, 1913-22.

Bult C. J., White O., Olsen G., J., Zhou L., Fleischmann R. D., Sutton G. G., Blake J. A., FitzGerald L. M., Clayton R. A., Gocayne J. D., Kerlavage A. R., Dougherty B. A., Tomb J. F., Adams M. D., Reich C. I., Overbeek R., Kirkness E. F., Weinstock K. G., Merrick J. M., Glodek A., Scott J. L., Geoghagen N. S., Venter J. C. (1996) Complete genome sequence of the methanogenic archaeon, Methanococcus jannaschii. Science, 273, 1058-1073.

De Barros Lopes, M., Rehman A., Gockowiak H., Heinrich A. J., Langridge P. and Henschke P. A. (2000) Fermentation properties of a wine yeast overexpressing the Saccharomyces glycerol 3-phosphate dehydrogenase (GPD2). Aust. J. Grape Wine Res. 6: 208-215.

Bely, M., Sablayrolles, J. M. and Barre, P. (1990). Automatic detection of assimilable nitrogen defiencies during alcoholic fermentation in oenological conditions. J. Ferm. Bioeng. 70, 246-252.

Bergmeyer, H. U. (1955). Zur messung von katalase aktivitäten. Biochem. Z. 327, 255-258.

Dequin, S. and Barre, P. (1994). Mixed lactic acid-alcoholic fermentation by Saccharomyces cerevisiae expressing the Lactobacillus casei L(+)-LDH. Biotechnology (NY) 12, 173-7.

Dequin, S. (2001) The potential of genetic engineering for improving brewing, wine-making and baking yeasts. AppI. Microbiol. Biotechnol. 56: 577-588.

Dequin, S., Salmon, J. M., Nguyen, H. V. and B. Blondin. (2003) Wine yeast's. In “Yeasts in Food, beneficial and detrimental aspects”, p 389-412. Eds T. Boekhout and V. Robert, B. Berhr's Verlag GmbH and Co Hamburg, Germany.

Dursun, G., Özer, A., Elibol, M. and Özer, D. (1999). Mass transfer characteristics of a fermentation broth in a reactor: co-current downflow contacting reactor. Process Biochemistry 34, 133-137.

Eglinton J M, Heinrich A J, Pollnitz A P, Langridge P, Henschke P A, de Barros Lopes M. (2002) Decreasing acetic acid accumulation by a glycerol overproducing strain of Saccharomyces cerevisiae by deleting the ALD6 aldehyde dehydrogenase gene. Yeast 19: 295-301.

Gonzalez, B., Francois, J. and Renaud, M. (1997). A rapid and reliable method for metabolite extraction in yeast using boiling buffered ethanol. Yeast 13, 1347-55.

Gonzalez, E., Fernandez, M. R., Larroy, C., Sola, L., Pericas, M. A., Pares, X. and Biosca, J. A. (2000). Characterization of a (2R,3R)-2,3-butanediol dehydrogenase as the Saccharomyces cerevisiae YAL060W gene product. Disruption and induction of the gene. J Biol Chem 275, 35876-85.

Hallsworth, J. E. (1998). Ethanol-Induced Water Stress in Yeast. Journal of Fermentation and Bioengineering 85, 125-137.

Himmelreich R, Hilbert H, Plagens H, Pirkl E, Li B C, Herrmann R (1996) Complete sequence analysis of the genome of the bacterium Mycoplasma pneumoniae. Nucleic Acids Res. 24:4420

Hoefnagel, M. H., Starrenburg, M. J., Martens, D. E., Hugenholtz, J., Kleerebezem, M., Van, S., I. I., Bongers, R., Westerhoff, H. V. and Snoep, J. L. (2002). Metabolic engineering of lactic acid bacteria, the combined approach: kinetic modelling, metabolic control and experimental analysis. Microbiology 148, 1003-13.

Holzer, H., Busch, D. and Kroger, H. (1958). [Enzymicoptic determination of reduced and oxidized triphosphopyridine nucleotides in presence of reduced and oxidized diphosphopyridine nucleotides.]. Hoppe Seylers Z Physiol Chem 313, 184-93.

Klingenberg, M. (1974). Nicotinamide-adenine dinucleotides (NAD, NADP, NADH, NADPH): spectrophotometric and fluorimetric methods in In H. U. Bergmeyer (ed.) (Ed), Methods of enzymatic analysis, pp. 2045-2059.

Koike K, Kobayashi T, Ito S, Saitoh M. (1985) Purification and characterization of NADH oxidase from a strain of Leuconostoc mesenteroides. J Biochem (Tokyo) 97,1279-88.

Liu, S. Q. and Pilone, G. J. (2000). An overview of formation and roles of acetaldehyde in winemaking with emphasis on microbiological implications. International Journal of Food Science & Technology 35, 49-61.

Lopez de Felipe, F. and Hugenholtz, J. (1999). Pyruvate flux distribution in NADH-oxidase-overproducing Lactococcus lactis strain as a function of culture conditions. FEMS Microbiol Lett 179, 461-6.

Lopez de Felipe, F. and Hugenholtz, J. (2001). Purification and characterisation of the water forming NADH-oxidase from Lactococcus lactis. International Dairy Journal 11, 37-44.

Lopez de Felipe, F., Kleerebezem, M., de Vos, W., M., and Hugenholtz, J. (1998). Cofactor engineering: a novel approach to metabolic engineering in Lactococcus lactis by controlled expression of NADH oxidase. J Bacteriol 180, 3804-8.

Lundquist, F. (1974). Acetaldehyd : Bestimmung Mit Aldehyd-dehydrogenase, Methods of Enzymatic Analysis, Academic Press, Inc., pp. 1509-1513.

Michnick, S., Roustan, J. L., Remize, F., Barre, P. and Dequin, S. (1997). Modulation of glycerol and ethanol yields during alcoholic fermentation in Saccharomyces cerevisiae strains overexpressed or disrupted for GPD1 encoding glycerol 3-phosphate dehydrogenase. Yeast 13, 783-93.

Malherbe, D. F., Du Toit, M., Cordero Otero, R. R., Van Rensburg, P. and Pretorius, I. S. (2003). Expression of the Aspergillus niger glucose oxidase gene in Saccharomyces cerevisiae and its potential applications in wine production. Appl Microbiol Biotechnol 61, 502-11.

Matsumoto J., Higushi M., Shimada M., Yamamoto Y., Kamio Y. (1996) Molecular cloning and sequence analysis of the gene encoding the H2O-forming NADH oxidase from Streptococcus mutans. Biosci. Biotech. Biochem., 60, 39-43.

Michnick, S., Roustan, J. L., Remize, F., Barre, P. and Dequin, S. (1997). Modulation of glycerol and ethanol yields during alcoholic fermentation in Saccharomyces cerevisiae strains overexpressed or disrupted for GPD1 encoding glycerol 3-phosphate dehydrogenase. Yeast 13, 783-93.

Minet, M., Dufour, M. E. and Lacroute, F. (1992). Complementation of Saccharomyces cerevisiae auxotrophic mutants by Arabidopsis thaliana cDNAs. Plant J 2, 417-22.

Mumberg, D., Muller, R. and Funk, M. (1995). Yeast vectors for the controlled expression of heterologous proteins in different genetic backgrounds. Gene 156, 119-22.

Nevoigt, E. and U. Stahl. 1996. Reduced pyruvate decarboxylase and increased glycerol-3-phosphate dehydrogenase [NAD+] levels enhance glycerol production in Saccharomyces cerevisiae. Yeast 12:1331-1337.

Nevoigt E, Pilger R, Mast-Gerlach E, Schmidt U, Freihammer S, Eschenbrenner M, Garbe L, Stahl U. (2002). Genetic engineering of brewing yeast to reduce the content of ethanol in beer. FEMS Yeast Res. 2:225-32.

Nissen, T. L., Schulze, U., Nielsen, J. and Villadsen, J. (1997). Flux distributions in anaerobic, glucose-limited continuous cultures of Saccharomyces cerevisiae. Microbiology 143, 203-218.

Peterson S. N., Hu P. C., Bott K. F., C. Hutchinson A. 3rd (1993) A survey of the Mycoplasma genitalium genome by using random sequencing. J. Bacteriol., 175, 7918-7930.

Remize, F., Roustan, J. L., Sablayrolles, J. M., Barre, P. and Dequin, S. (1999) Glycerol overproduction by engineered Saccharomyces cerevisiae wine yeast strains leads to substantial changes in by-product formation and to a stimulation of fermentation rate in stationary phase. Appl. Environ. Microbiol. 65: 143-149.

Remize, F., Andrieu, E. and Dequin, S. (2000) Engineering of the pyruvate dehydrogenase by-pass in S. cerevisiae—Role of the cytosolic Mg²⁺ and mitochondrial K⁺ acetaldehyde dehydrogenases Ald6p and Ald4p in acetate formation during alcoholic fermentation. Appl. Environ. Microbiol. 66: 3151-3159.

Remize, F., Barnavon, L. and Dequin, S. (2001) Glycerol export and glycerol 3-phosphate dehydrogenase, but not glycerol phosphatase, are rate limiting steps for glycerol production in Saccharomyces cerevisiae. Metab. Eng. 3: 301-312.

Riebel B. R., Gibbs P. R., Wellborn W. B., Bommarius A. S. (2002) Cofactor Regeneration of NAD⁺ from NADH: Novel water-forming NADH oxidases. Adv. Synth. Catal. 3444, 1156-1168.

Ross R. P., Claiborne A. (1991) Cloning, sequence and overexpression of NADH peroxidase from Streptococcus faecalis 10C1. Structural relationship with the flavoprotein disulfide reductases. J. Mol. Biol., 221, 857-871.

Ross R. P., Claiborne A. (1992) Molecular cloning and analysis of the gene encoding the NADH oxidase from Streptococcus faecalis 10C1. Comparison with NADH peroxidase and the flavoprotein disulfide reductases. J. Mol. Biol., 227, 65 8-671.

Roustan, J. L. and Sablayrolles, J. M. (2002). Impact of the addition of electron acceptors on the by-products of alcoholic fermentation. Enzyme and microbial technology 31, 142-152.

Sablayrolles, J. and Barre, P. (1986). Evolution de la solubilité de l'oxygéne au cours de la fermentation alcoolique d'un moût de raisin. Etude sur milieu modéle. Sciences des aliments 6, 177-184.

Sambrook, J., Fritsh, E. F. and Maniatis, T. (1989). Molecular Cloning: A Laboratory Manual, 2nd edn. Cold Spring Harbor Laboratory press, Cold spring Harborg.

Saint-Prix, F., Bonquist, L. and Dequin, S. (2004). Functional analysis of the ALD gene family of Saccharomyces cerevisiae during anaerobic growth on glucose: the NADP+-dependent Ald6p and Ald5p isoforms play a major role in acetate formation. Microbiology 150, 2209-20.

Schiestl, R. H. and Gietz, R. D. (1989). High efficiency transformation of intact yeast cells using single stranded nucleic acids as a carrier. Curr Genet 16, 339-46.

Teusink, B., Passarge, J., Reijenga, C. A., Esgalhado, E., van der Weijden, C. C., Schepper, M., Walsh, M. C., Bakker, B. M., van Dam, K., Westerhoff, H. V. and Snoep, J. L. (2000). Can yeast glycolysis be understood in terms of in vitro kinetics of the constituent enzymes? Testing biochemistry. Eur J Biochem 267, 5313-29.

Vernet, T., Dignard, D. and Thomas, D. Y. (1987). A family of yeast expression vectors containing the phage f1 intergenic region. Gene 52, 225-33.

1. A transformed yeast strain belonging to the genus Saccharomyces which comprises a heterologous nucleic acid coding for a water-producing NADH oxidase.

2. The transformed yeast strain according to claim 1, wherein the heterologous nucleic acid coding for a water-producing NADH oxidase is integrated into the genome of said yeast.

3. The transformed yeast strain according to claim 1, wherein said strain of the genus Saccharomyces is a Saccharomvces cerevisiae strain.

4. The transformed yeast strain according to claim 1, wherein said water-producing NADH oxidase is of bacterial origin.

5. The transformed yeast strain according to claim 1, wherein said heterologous nucleic acid coding for a water-producing NADH oxidase comprises a sequence selected from the group constituted of the sequences SEQ ID No.1, SEQ ID No.2, SEQ ID No.3, SEQ ID No.4, SEQ ID No.5 and SEQ ID No.6.

6. A method for the preparation of a transformed yeast strain belonging to the genus Saccharomyces which has, during alcoholic fermentation, a reduced production yield of ethanol as compared with the wild-type Saccharomyces strain, said process comprising a step consisting of introducing at least one heterologous nucleic acid coding for a water-producing NADH oxidase into a yeast strain of the genus Saccharomyces.

7. The method according to claim 6, wherein said heterologous nucleic acid coding for a water-producing NADH oxidase is introduced into the genome of said yeast strain of the genus Saccharomyces.

8. The method according to claim 6, wherein said strain of the genus Saccharomyces is a Saccharomvces cerevisiae strain.

9. The method according to claim 6, wherein said water-producing NADH oxidase is of bacterial origin.

10. The method according claim 6, wherein said heterologous nucleic acid coding for a water-producing NADH oxidase comprises a sequence selected from the group constituted of the sequences SEQ ID No.1, SEQ ID No.2, SEQ ID No.3, SEQ ID No.4, SEQ ID No.5 and SEQ ID No.6.

11. The method according to claim 6, wherein the production yield of ethanol of the transformed strain of the genus Saccharomyces is reduced by 1° as compared with the wild-type strain of the genus Saccharomyces.

12. An expression cassette comprising a sequence coding for a water-producing NADH oxidase of bacterial origin in association with sequences for regulation of the expression of said sequence coding for a water-producing NADH oxidase in the yeast.

13. A vector comprising the expression cassette according to claim 12.

14. A method of conducting alcoholic fermentation which comprises the use of a transformed yeast strain belonging to the genus Saccharomyces according to claim 1.

15. The method according to claim 14, wherein alcoholic fermentation is carried out during preparation of a fermented drink or in bread-making.

16. A fermentation process comprising steps consisting of:
a) inoculating a medium having a high sugar content with at least one transformed yeast strain belonging to the genus Saccharomyces according to claim 1;
b) cultivating said transformed Saccharomyces yeast strain; and
c) allowing the fermentation to take place in order to convert the sugars into alcohol.

17. The fermentation process according to claim 16, said process being a wine-making process and the medium having a high sugar content being a grape must.

18. The wine-making process according to claim 17, wherein said at least one transformed Saccharomyces yeast strain is initially cultivated under anaerobic conditions and is then cultivated under aerobic conditions when substantially the totality of the nitrogen of the grape must has been consumed.

19. A method of regenerating NAD+ from NADH which comprises the use of a transformed yeast strain belonging to the genus Saccharomyces according to claim 1.
